SEQUENCE LISTING (7 sequences)

SEQ ID NO: 1 - DNA, 29 bp, synthetic construct
  source 1..29: mol_type = other DNA
  modified_base 25..29: mod_base = OTHER; note = phosphorothioate linkage
  modified_base 29: mod_base = OTHER; note = 3' dideoxycytosine
gatcggaagagcacgtctgaactccagtn

SEQ ID NO: 2 - DNA, 33 bp, synthetic construct
  source 1..33: mol_type = other DNA
  modified_base 30..33: mod_base = OTHER; note = phosphorothioate linkage
  modified_base 31..32: mod_base = OTHER; note = inosine
acactctttccctacacgactcttccgatcnna

SEQ ID NO: 3 - DNA, 73 bp, synthetic construct
  source 1..73: mol_type = other DNA
gaatctggcggacccggcttaccgccgccgtcgcatcatcaaacggcgtcagccgatctgctcagttgcatcc

SEQ ID NO: 4 - DNA, 73 bp, synthetic construct
  source 1..73: mol_type = other DNA
gagcagatcggctgacgccgtttgatgatgcgacggcggcggtaagccgggtccgccagattcggtgctctcc

SEQ ID NO: 5 - DNA, 90 bp, synthetic construct
  source 1..90: mol_type = other DNA
ggagctgaagccatccgggcactggccacacacgcgtggttggcagggcttggaaaaagcatttgtaaagaaagaaatcgaccgggctcc

SEQ ID NO: 6 - DNA, 90 bp, synthetic construct
  source 1..90: mol_type = other DNA
ggagcccggtcgatttctttctttacaaatgctttttccaagccctgccaaccacgcgtgtgtggccagtgcccggatggcttcagctcc

SEQ ID NO: 7 - DNA, 72 bp, synthetic construct
  source 1..72: mol_type = other DNA
gttatcaggtcatgaacggcatcgccgtgcccgttcaatctgtctgatgtatccatattggatctggtatcc

(19) United States
(12) Patent Application Publication
(10) Pub. No.: US 2026/0201440 A1
(43) Pub. Date: Jul. 16, 2026

(54) OLIGONUCLEOTIDES AND METHODS FOR CAPTURING SINGLE-STRANDED TEMPLATES AND/OR TEMPLATES WITH 3' OVERHANGS

(71) Applicant: Foundation Medicine, Inc., Boston, MA (US)

(72) Inventors: James YU, San Diego, CA (US); Nicole LAMBERT, San Diego, CA (US)

(73) Assignee: Foundation Medicine, Inc., Boston, MA (US)

(21) Appl. No.: 19/137,409

(22) PCT Filed: Dec. 13, 2023
(86) PCT No.: PCT/US2023/083916

§ 371 Date: Jun. 10, 2025

Related U.S. Application Data
(60) Provisional application No. 63/435,002, filed on Dec. 23, 2022.

(51) Int. Cl. C12Q 1/6806; C12Q 1/48; C12Q 1/6855; C12Q 1/686; C12Q 1/6869; C12Q 1/6876

(52) U.S. Cl. CPC C12Q 1/6806; C12Q 1/48; C12Q 1/6855; C12Q 1/686; C12Q 1/6869; C12Q 1/6876; C12Q 2600/154; C12Q 2600/156; C12Y 207/07; C12Y 605/01001

(57) ABSTRACT

Provided herein are oligonucleotides, kits, and methods for appending an oligonucleotide adapter to a single-stranded DNA (ssDNA) fragment and/or double-stranded DNA (dsDNA) fragment comprising 3′ overhang(s). In some embodiments, the adapter comprises a first strand comprising, from 5′ to 3′: a first portion comprising one or more nucleotides, and a second portion comprising three or more consecutive nucleotides joined by phosphorothioate linkages followed by a 3′ terminal dideoxynucleotide; and a second strand comprising, from 5′ to 3′: a first portion comprising one or more nucleotides, and a second portion comprising at least two deoxyinosine nucleotides (diTPs) joined by a phosphorothioate linkage followed by a 3′ terminal deoxynucleotide.

### CROSS-REFERENCE TO RELATED APPLICATIONS

This application claims the priority benefit of U.S. Provisional Application No. 63/435,002, filed Dec. 23, 2022, which is hereby incorporated by reference in its entirety.

### REFERENCE TO AN ELECTRONIC SEQUENCE LISTING

The contents of the electronic sequence listing (197102013140seqlist.xml; Size: 9,745 bytes; and Date of Creation: Dec. 13, 2023) are herein incorporated by reference in their entirety.

### FIELD

Provided herein are oligonucleotides, kits, and methods for appending an oligonucleotide adapter to a single-stranded DNA (ssDNA) fragment and/or double-stranded DNA (dsDNA) fragment comprising 3′ overhang(s).

### BACKGROUND

Tremendous strides have been made in genome sequencing technologies since sequencing the human genome in 2003, leading to an increase in the number and diversity of sequenced genomes and a wealth of information related to basic biology and disease. Typically, high-throughput sequencing approaches such as next-generation sequencing (NGS) require that the nucleic acids (e.g., DNA) to be sequenced have instrument-specific DNA sequences or adapters appended onto each end of the molecule prior to analysis. This process is known as NGS library preparation. However, typical library preparation methods do not capture every DNA molecule in its entirety. For example, 3′ overhangs in otherwise double-stranded DNA (dsDNA) molecules or fragments are typically degraded (e.g., enzymatically), leaving a blunt-ended dsDNA molecule or fragment. See, e.g., Troll, C. J. et al. (2019) BMC Genomics 20 (1): 1023. Single-stranded DNA (ssDNA) molecules are also excluded from typical NGS library preparation.

Molecules such as ssDNA or dsDNA fragments with 3′ overhangs that are excluded by standard NGS library preparation methods could potentially contain useful information, e.g., as biomarkers. Analysis of the patterns of DNA fragmentation (i.e., fragmentomics), such as the non-random starts/ends of cell-free DNA (cfDNA), could provide useful diagnostic and/or conceptual information, as these patterns are thought to arise from nucleosome positioning and/or chromatin state/features. See, e.g., Ivanov, M. et al. (2015) BMC Genomics 16 (Suppl 13): S1.

For example, regions of the genome not occupied by nucleosomes are thought to be more susceptible to nucleases. Therefore, mapping the ends of ssDNA and/or 3′ overhangs of dsDNA could be used to infer nucleosome position or other chromatin features. However, current fragmentomics approaches can rely only on 5′ ends of DNA fragments, since 3′ ends cannot be accurately mapped due to aforementioned degradation during library preparation. See, e.g., Jiang, P. et al. (2020) Cancer Discov. 10 (5): 664-673.

In addition to fragmentomics, because cfDNA concentrations in samples are typically low, expanding the pool of polynucleotides for analysis to ssDNA and/or dsDNA fragments with 3′ overhangs could offer orthogonal tumor signals (e.g., dsDNA for variant calling and/or ssDNA/dsDNA fragments with 3′ overhangs for fragmentomics). Capturing all forms of DNA without any degradation may uncover potential new cancer biology/biomarkers. For instance, quantifying the proportion of single- to double-stranded DNA cannot be performed using current library preparation methods.

Some commercial NGS library preparation kits allow for retaining ssDNA and/or dsDNA with 3′ overhang(s). For example, the xGen™ NGS kit (IDT) allows for capturing ssDNA and dsDNA without degradation of 3′ overhangs. However, this method uses an adaptase that results in addition of a variable number of nucleotides onto the 3′ end, making the precise mapping of the 3′ termini difficult. In addition, the SRSLY® NGS Library Prep Kit (Claret Biosciences) also allows for retaining ssDNA and/or dsDNA with 3′ overhang(s), but, like the xGen™ NGS kit, includes an initial heating step to denature all double-stranded molecules into single strands, thereby foreclosing the ability to retain duplex information, e.g., for double-stranded error correction (thus potentially reducing NGS assay sensitivity). Such ssDNA library preparation methods can lead to lower unique molecular depth and/or higher rates of chimeric reads, compared to dsDNA library preparation. U.S. PG Pub. No. 20210123097A1 describes the use of randomers and gap-filling by Klenow DNA polymerase, but this polymerase has strand displacement activity, which can lead to resynthesis of DNA during 3′ overhang capture and end repair, confounding any analysis that would require retention of native bases (e.g., methylation sequencing). Randomers can also increase cost and anneal to each other, forming adapter dimers that reduce the number of usable sequencing reads. U.S. Pat. No. 10,487,358 describes a sequential workflow where dsDNA library prep is followed by ssDNA library prep. However, this would also confound any analysis that would require retention of native bases (e.g., methylation sequencing), and ssDNA library prep could also lead to lower unique molecular depth and/or potentially high rates of chimeric reads.

Therefore, the need exists for methods that allow for capture and NGS library preparation of ssDNA and dsDNA fragments with 3′ overhangs while retaining duplex information and enabling precise mapping of 3′ overhangs and their termini.

All references cited herein, including patent applications and publications, are incorporated by reference in their entirety.

### SUMMARY OF THE INVENTION

The present disclosure provides, inter alia, oligonucleotides (as well as kits and methods related thereto) for capturing single-stranded templates and/or templates (e.g., double-stranded templates) with one or more 3′ overhang(s). Advantageously, these methods preserve duplex dsDNA information, allowing for double-stranded error correction, while also capturing single-stranded molecules. In addition, these methods allow for precise mapping of 3′ overhangs and their ends, providing detailed fragmentomic information. They are also amenable to subsequent standard library preparation of dsDNA with blunt ends and/or 5′ overhangs, allowing the ability to sequence blunt-ended dsDNA fragments, dsDNA fragments with 5′ and/or 3′ overhang(s), and ssDNA fragments in a unified workflow.

In one aspect, provided herein are oligonucleotides comprising: a) a first strand that comprises, from 5′ to 3′: i) a first portion comprising one or more nucleotides, and ii) a second portion comprising three or more consecutive nucleotides joined by phosphorothioate linkages followed by a 3′ terminal dideoxynucleotide (ddNTP); and b) a second strand that comprises, from 5′ to 3′: i) a first portion comprising one or more nucleotides, wherein the first portion of the second strand is capable of hybridizing with the first portion of the first strand; and ii) a second portion comprising at least two deoxyinosine nucleotides (dITPs) joined by a phosphorothioate linkage followed by a 3′ terminal deoxynucleotide; wherein upon hybridization of the first portion of the first strand with the first portion of the second strand, the second portion of the second strand forms a 3′ overhang.

In another aspect, provided herein are kits comprising the oligonucleotides according to any one of the embodiments disclosed herein. In some embodiments, the kits comprise a first oligonucleotide according to any one of the embodiments described herein, wherein the 3′ terminal deoxynucleotide of the second strand is a deoxyadenosine trisphosphate (dATP); a second oligonucleotide according to any one of the embodiments described herein, wherein the 3′ terminal deoxynucleotide of the second strand is a deoxycytidine trisphosphate (dCTP); a third oligonucleotide according to any one of the embodiments described herein, wherein the 3′ terminal deoxynucleotide of the second strand is a deoxyguanosine trisphosphate (dGTP); and a fourth oligonucleotide according to any one of the embodiments described herein, wherein the 3′ terminal deoxynucleotide of the second strand is a deoxythymidine trisphosphate (dTTP).

In another aspect, provided herein is a method of appending an oligonucleotide adapter to a single-stranded DNA (ssDNA) fragment or double-stranded DNA (dsDNA) fragment comprising 3′ overhang(s), comprising: a) contacting an oligonucleotide adapter with a plurality of ssDNA fragments and/or dsDNA fragments comprising 3′ overhang(s) under conditions suitable for hybridization of the adapter with a ssDNA or dsDNA fragment of the plurality, wherein the adapter comprises: a first strand comprising, from 5′ to 3′: a first portion comprising one or more nucleotides, and a second portion comprising three or more consecutive nucleotides joined by phosphorothioate linkages followed by a 3′ terminal dideoxynucleotide; and a second strand comprising, from 5′ to 3′: a first portion comprising one or more nucleotides, and a second portion comprising at least two deoxyinosine nucleotides (dITPs) joined by a phosphorothioate linkage followed by a 3′ terminal deoxynucleotide; wherein the first portion of the second strand of the adapter hybridizes with the first portion of the first strand of the adapter; and wherein the second portion of the second strand of the adapter hybridizes with a 3′ end of the ssDNA fragment of the plurality or a 3′ overhang of the dsDNA fragment of the plurality; b) incubating the hybridized adapter and ssDNA or dsDNA fragment with a DNA ligase under conditions suitable for sealing a nick between a 3′ end of the ssDNA fragment or overhang of the dsDNA fragment and the 5′ end of the first portion of the first strand of the adapter; and c) incubating the hybridized adapter and fragment with a DNA polymerase and nucleotides under conditions suitable for the polymerase to catalyze strand extension from the 3′ terminal deoxynucleotide of the second strand of the adapter using the ssDNA fragment or overhang of the dsDNA fragment as a template, wherein the DNA polymerase lacks strand displacement activity, thereby appending the adapter onto the fragment.

In some embodiments according to any of the embodiments described herein, the 3′ terminal deoxynucleotide of the second strand is a deoxyadenosine trisphosphate (dATP), deoxycytidine triphosphate (dCTP), deoxyguanosine triphosphate (dGTP), or deoxythymidine triphosphate (dTTP). In some embodiments, the one or more nucleotides of the first portions of the first and second strands are deoxynucleotide triphosphate (dNTP) nucleotides. In some embodiments, the first portion of the first strand and the first portion of the second strand each independently comprise 10 or more nucleotides. In some embodiments, the second portion of the second strand comprises at least two consecutive dITPs joined by a phosphorothioate linkage followed by the 3′ terminal deoxynucleotide. In some embodiments, the second portion of the second strand comprises two consecutive dITPs joined by a phosphorothioate linkage followed by the 3′ terminal deoxynucleotide. In some embodiments, the second strand further comprises a third portion comprising one or more nucleotides, wherein the third portion is 5′ of the first portion, and wherein, upon hybridization of the first portion of the first strand with the first portion of the second strand, the third portion of the second strand does not hybridize with the second portion of the first strand. In some embodiments, the first strand comprises the sequence of SEQ ID NO:1 and/or the second strand comprises the sequence of SEQ ID NO:2.

In some embodiments according to any of the embodiments described herein, incubating the hybridized adapter and ssDNA or dsDNA fragment comprises incubating the hybridized adapter and ssDNA or dsDNA fragment with the DNA ligase and a polynucleotide kinase (PNK) under conditions suitable for sealing a nick between a 3′ end of the ssDNA fragment or overhang of the dsDNA fragment and the 5′ end of the first portion of the first strand of the adapter. In some embodiments, the adapter is hybridized with a dsDNA fragment comprising a 3′ overhang, and the method further comprises, after strand extension in c), incubating the hybridized adapter and fragment with a DNA ligase under conditions suitable for sealing a nick between the strand synthesized in c) and a 5′ end of the dsDNA fragment. In some embodiments, the methods further comprise, after strand extension in c), incubating the hybridized adapter and fragment with the DNA ligase and a PNK under conditions suitable for sealing a nick between the strand synthesized in c) and a 5′ end of the dsDNA fragment. In some embodiments, the DNA polymerase is a T4 DNA polymerase. In some embodiments, the DNA ligase is a T3 DNA ligase, T4 DNA ligase, or Taq DNA ligase. In some embodiments, the plurality of ssDNA fragments and/or dsDNA fragments comprising 3′ overhang(s) are contacted in a) with a plurality of adapters that comprises a first adapter with deoxyadenosine trisphosphate (dATP) as the 3′ terminal deoxynucleotide of the second strand, a second adapter with deoxycytidine triphosphate (dCTP) as the 3′ terminal deoxynucleotide of the second strand, a third adapter with deoxyguanosine triphosphate (dGTP) as the 3′ terminal deoxynucleotide of the second strand, and a fourth adapter with deoxythymidine triphosphate (dTTP) as the 3′ terminal deoxynucleotide of the second strand. In some embodiments, the nucleotides of c) comprise at least one modified nucleotide. In some embodiments, the modified nucleotide is 8-oxoguanine, 5-methylcytosine (5mC), or inosine. In some embodiments, the methods further comprise, e.g., prior to a), isolating the plurality of ssDNA fragments and/or dsDNA fragments comprising 3′ overhang(s) from a sample. In some embodiments, the sample comprises cell-free DNA (cfDNA), circulating cell-free DNA (ccfDNA), or circulating tumor DNA (ctDNA). In some embodiments, the sample comprises fluid, cells, or tissue. In some embodiments, the sample comprises tumor cells and/or tumor nucleic acids. In some embodiments, the sample further comprises blunt-ended dsDNA fragments and/or dsDNA fragments comprising 5′ overhang(s), and wherein the method further comprises, prior to a), isolating the blunt-ended dsDNA fragments and/or dsDNA fragments comprising 5′ overhang(s) with the plurality of ssDNA fragments and/or dsDNA fragments comprising 3′ overhang(s). In some embodiments, the methods further comprise, e.g., after strand extension in c), (i) subjecting the blunt-ended dsDNA fragments and/or dsDNA fragments comprising 5′ overhang(s) to end repair; (ii) subjecting the end-repaired dsDNA fragments to 3′ adenylation; and/or (iii) subjecting the 3′ adenylated dsDNA fragments to adapter ligation. In some embodiments, (i)-(iii) are performed in the presence of the product(s) of strand extension from c). In some embodiments, the methods further comprise, e.g., after strand extension in c), isolating the product(s) of strand extension from free oligonucleotide adapters. In some embodiments, the methods further comprise, e.g., after strand extension in c), subjecting the product(s) of strand extension to polymerase chain reaction (PCR) amplification. In some embodiments, the methods further comprise, e.g., after strand extension in c), subjecting the product(s) of strand extension or PCR amplicon(s) thereof to sequencing or methylation sequencing. In some embodiments, the nucleotides of c) comprise 8-oxoguanine, wherein the method further comprises subjecting the product(s) of strand extension to polymerase chain reaction (PCR) amplification after strand extension in c), and wherein the sequencing comprises identifying a guanine→thymine mutation introduced by PCR corresponding to a position of an adenine nucleotide base-paired with an 8-oxoguanine during strand extension.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1D illustrate an exemplary oligonucleotide (e.g., 3′ end capture adapter) of the present disclosure and some of its attributes of interest. FIG. 1A shows the adapter, highlighting exemplary features. The first strand (top, with 5′ to 3′ direction shown left to right) includes, from 5′ to 3′, a first portion and a second portion, with the second portion ending in 3 nucleotides joined by phosphorothioate bonds followed by a 3′ terminal dideoxynucleotide (ddNTP). The second strand (bottom, with 5′ to 3′ direction shown right to left) includes, from 5′ to 3′, a portion (e.g., third portion of the present disclosure) of non-complementarity with the second portion of the first strand, a first portion that hybridizes with the first portion of the first strand, and a second portion with a 3′ overhang having 2 deoxyinosine nucleotides (dITPs) joined by phosphorothioate bonds followed by a 3′ terminal deoxynucleotide (dNTP). FIG. 1B illustrates that formation of adapter dimers is prevented by inclusion of dITPs in the 3′ overhang, since inosine-inosine basepairing is highly unstable (I: dITP; N: dNTP). FIG. 1C illustrates that adapter concatenation is prevented by the inclusion of the 3′ terminal ddNTP in the first strand, which prevents elongation/ligation from this end. FIG. 1D illustrates that rogue 3′ priming is also prevented by the inclusion of the 3′ terminal ddNTP in the first strand, which prevents priming of template molecules from this end.

FIGS. 2A-2C illustrate use of the exemplary oligonucleotide (e.g., 3′ end capture adapter) shown in FIG. 1A in capturing a double-stranded (ds) template with 3′ overhangs. In FIG. 2A, one adapter hybridizes to each overhang, with the overhanging dITPs and dNTP of the second strand of each adapter hybridizing with the respective 3′ ends of the template, resulting in an annealing of 3 base pairs between each adapter and the template. In FIG. 2B, nicks between the 5′ end of the first strand of each adapter and the 3′ end of the template overhang are sealed (e.g., using a DNA ligase and polynucleotide kinase, PNK). In FIG. 2C, DNA polymerase extends from the 3′ terminal dNTP of the second strand of each adapter using the template overhang as a template (direction marked by arrows). One or more modified nucleotides can be used to mark this newly added sequence (asterisks). Since the DNA polymerase has no strand displacement activity, it leaves a nick (carets), which can be sealed (e.g., using a DNA ligase and PNK).

FIG. 2D shows how standard library preparation methods deal with overhangs by filling in 5′ overhangs (right) and degrading 3′ overhangs (left) to create blunt ends prior to adapter ligation. In contrast to the protocol shown in FIGS. 2A-2C, this standard protocol removes information about the sequence and precise position of 3′ overhangs in ds fragments.

FIG. 3 shows processing of a ds template with one blunt end (left) and one 3′ overhang (right) using traditional adapters and exemplary oligonucleotides of the present disclosure shown in FIG. 1A (top). The 3′ overhang is processed as shown in FIGS. 2A-2C, after which the resulting molecule can be subjected to standard library preparation (e.g., end repair, A-tailing, adapter ligation) using standard adapters without disrupting the product of FIGS. 2A-2C. If the 3′ overhang molecules were in a mixture with blunt ended dsDNA and/or dsDNA with only 5′ overhangs, these can be processed as well as part of the standard library preparation. The end result (bottom) is a template molecule with a traditional adapter ligated to the blunt end (left) and the former 3′ overhang sequence captured by the exemplary oligonucleotide of the present disclosure now appended to that end (right).

FIGS. 4A & 4B illustrate use of the exemplary oligonucleotide (e.g., 3′ end capture adapter) shown in FIG. 1A in capturing a single-stranded (ss) template. In FIG. 4A, the adapter hybridizes to the 3′ end of the ss template, with the overhanging dITPs and dNTP of the second strand of the adapter hybridizing with the 3′ end of the template, resulting in an annealing of 3 base pairs between the adapter and template. The nick between the 5′ end of the first strand of the adapter and the 3′ end of the template is further sealed (e.g., using a DNA ligase and PNK). In FIG. 4B, DNA polymerase extends from the 3′ terminal dNTP of the second strand of the adapter based on the ss template (top). If desired, the blunt end of the template and resulting strand can be ligated to a traditional adapter using standard library prep methods (e.g., after end repair and A-tailing; bottom left) after capturing the other end of the ssDNA template using the 3′ end capture adapter now appended to the template (bottom right).

FIGS. 5A-5C show the results of testing traditional adapters and 3′ end capture adapters on various templates: an 83 base pair (bp) dsDNA fragment with 10 bp 3′ overhangs, a blunt-ended 90 bp dsDNA fragment with no overhangs, and a 72 bp ssDNA fragment. FIG. 5A shows the results of processing the three templates using 3′ end capture adapters as described in Example 1, quantifying products, and resolving product size via electrophoresis. 3′ end capture adapters gave a single peak corresponding to the 3′ overhang template, showing selectivity for 3′ overhangs. FIG. 5B shows the results of processing the three templates using traditional adapters as described in Example 1, quantifying products, and resolving product size via electrophoresis. Traditional adapters captured both dsDNA fragments (blunt-ended and with 3′ overhangs). FIG. 5C shows only the peaks corresponding to the 3′ overhang templates from FIGS. 5A & 5B. The peak obtained from traditional adapters was left-shifted compared to the peak obtained from 3′ end capture adapters. This suggests a smaller product, which is consistent with traditional adapters leading to degradation of 3′ overhangs, while 3′ end capture adapters preserving the 3′ overhangs.

FIGS. 6A-6D show the adapters and templates used in the experiments shown in FIGS. 5A-5C. FIG. 6A shows the sequence of the 3′ end capture adapter, with first strand (top, with 5′ to 3′ direction shown left to right) having the sequence of SEQ ID NO:1, and the second strand (bottom, with 5′ to 3′ direction shown right to left) having the sequence of SEQ ID NO:2. FIG. 6B shows the sequence of the dsDNA fragment with 3′ overhang (shown are SEQ ID Nos: 3 & 4 for top and bottom strands, respectively). FIG. 6C shows the sequence of the blunt-ended dsDNA fragment (shown are SEQ ID Nos: 5 & 6 for top and bottom strands, respectively). FIG. 6D shows the sequence of the ssDNA fragment (SEQ ID No: 7).

### DETAILED DESCRIPTION

The present disclosure relates generally to oligonucleotides (as well as kits and methods of use thereof) able to act as adapters and allow capturing of single-stranded templates, as well as double-stranded templates with 3′ overhangs. Advantageously, these oligonucleotides, kits, and methods allow for capture and library preparation of such templates while preserving 3′ overhangs, which are typically degraded in standard library preparation methods, and allowing the capture of single-stranded templates, which are typically not captured in standard library preparation methods. Preserving 3′ overhangs allows these sequences to be analyzed and their precise ends to be mapped, further suggesting utility in fragmentomics approaches and analysis of cfDNA. The oligonucleotides, kits, and methods are also compatible with subsequent standard library preparation methods, e.g., for capturing blunt-ended double-stranded templates and double-stranded templates with 5′ overhangs. This allows for analysis, library preparation, and/or sequencing all of these templates, thus widening the types of nucleic acid templates amenable to NGS analysis.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. De Vita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside.

One or more phosphodiester linkages may be replaced by alternative linking groups. These alternative linking groups include, but are not limited to, embodiments wherein phosphate is replaced by P(0)S (“thioate”), P(S) S (“dithioate”), “(0)NR₂ (“amidate”), P(0)R, P(0)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “detection” includes any means of detecting, including direct and indirect detection.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as cytosine analogs resistant to cytosine conversion, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

The term “methylation” is used herein to refer to presence of a methyl group at the C5 position of a cytosine nucleotide within DNA nucleic acids (unless context indicates otherwise). This term includes 5-methylcytosine (5mC) as well as cytosine nucleotides in which the methyl group is further modified, such as 5-hydroxymethylcytosine (5hmC). This term also includes DNA nucleic acids that have been subjected to chemical or enzymatic conversion of nucleotides, such as conversion that deaminates unmodified cytosines to uracil.

The term “aberrant methylation” is used herein to refer to a pattern of methylation that is not typically present in a normal tissue. For example, the term can refer to increased methylation at a site that is not normally methylated in a normal tissue, or decreased methylation at a site that is normally methylated in a normal tissue. In some embodiments, nucleic acids derived from a cancer cell (e.g., cancer nucleic acids) are characterized by aberrant methylation when their pattern and/or amount of methylation at one or more genomic loci differs from what is normally present at the corresponding locus/loci in a particular type of tissue.

An “article of manufacture” is any manufacture (e.g., a package or container) or kit comprising at least one reagent, e.g., an oligonucleotide adapter described herein. In certain embodiments, the manufacture or kit is promoted, distributed, or sold as a unit for performing the methods described herein. In some embodiments, the article of manufacture or kit comprises four different oligonucleotide adapters described herein, each having a different 3′ terminal dNTP of the second strand (e.g., A, C, G, and T).

### III. Oligonucleotides, Kits, and Methods

Certain aspects of the present disclosure relate to oligonucleotides, e.g., oligonucleotide adapters. In some embodiments, the oligonucleotides comprise a first strand and a second strand.

In some embodiments, the first strand comprises (from 5′ to 3′): a first portion comprising one or more nucleotides, and a second portion comprising three or more nucleotides joined by phosphorothioate linkages followed by a 3′ terminal dideoxynucleotide (ddNTP). In some embodiments, the second portion comprises three or more consecutive nucleotides joined by phosphorothioate linkages followed by a 3′ terminal dideoxynucleotide (ddNTP). In some embodiments, the second portion comprises three consecutive nucleotides joined by phosphorothioate linkages followed by a 3′ terminal dideoxynucleotide (ddNTP).

In some embodiments, the second portion of the first strand further comprises one or more index primer binding sites. In some embodiments, the second portion of the first strand further comprises one or more barcoding sequence(s). Advantageously, the first strand of the adapter can act as a traditional adapter during downstream library prep methods. Exemplary adapter sequences and components are known in the art; see, e.g., NEBNext® Ultra™ II Y adapters (NEB) and xGen™ Stubby Adapters (IDT).

In some embodiments, the second strand comprises (from 5′ to 3′): a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal deoxynucleotide. In some embodiments, the second portion of the second strand comprises at least two consecutive dITPs joined by a phosphorothioate linkage followed by the 3′ terminal deoxynucleotide. In some embodiments, the second portion of the second strand comprises two consecutive dITPs joined by a phosphorothioate linkage followed by the 3′ terminal deoxynucleotide. In some embodiments, upon hybridization of the first portion of the first strand with the first portion of the second strand, the second portion of the second strand forms a 3′ overhang (e.g., comprising the at least two dITPs joined by a phosphorothioate linkage and a 3′ terminal deoxynucleotide). In some embodiments, the first portion of the second strand is at least partially complementary to the first portion of the first strand. In some embodiments, the first portion of the second strand is a reverse complement of the first portion of the first strand.

In some embodiments, the 3′ terminal deoxynucleotide of the second strand is a deoxyadenosine trisphosphate (dATP), deoxycytidine triphosphate (dCTP), deoxyguanosine triphosphate (dGTP), or deoxythymidine triphosphate (dTTP).

In some embodiments, the second strand further comprises a third portion comprising one or more nucleotides, wherein the third portion is 5′ of the first portion. In some embodiments, upon hybridization of the first portion of the first strand with the first portion of the second strand, the third portion of the second strand does not hybridize with the second portion of the first strand. In some embodiments, the third portion of the second strand is not complementary to the second portion of the first strand. In some embodiments, the third portion of the second strand is incapable of hybridizing to the second portion of the first strand. In some embodiments, the second strand comprises (from 5′ to 3′): a third portion comprising one or more nucleotides, wherein the third portion does not hybridize with the second portion of the first strand; a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal deoxynucleotide.

In some embodiments, the third portion of the second strand further comprises one or more index primer binding sites. In some embodiments, the third portion of the second strand further comprises one or more barcoding sequence(s) or identifying sequences. Advantageously, this allows the second strand of the adapter to act as a traditional adapter during downstream library prep methods. Exemplary adapter sequences and components are known in the art; see, e.g., NEBNext® Ultra™ II Y adapters (NEB) and xGen™ Stubby Adapters (IDT). In some embodiments, the third portion of the second strand comprises different sequence(s) and/or index primer binding sites as compared to those of the second portion of the first strand, allowing for duplex sequencing and double-stranded error correction approaches (see, e.g., Schmitt, M. W. et al. (2012) Proc. Natl. Acad. Sci. USA 109 (36): 14508-14513).

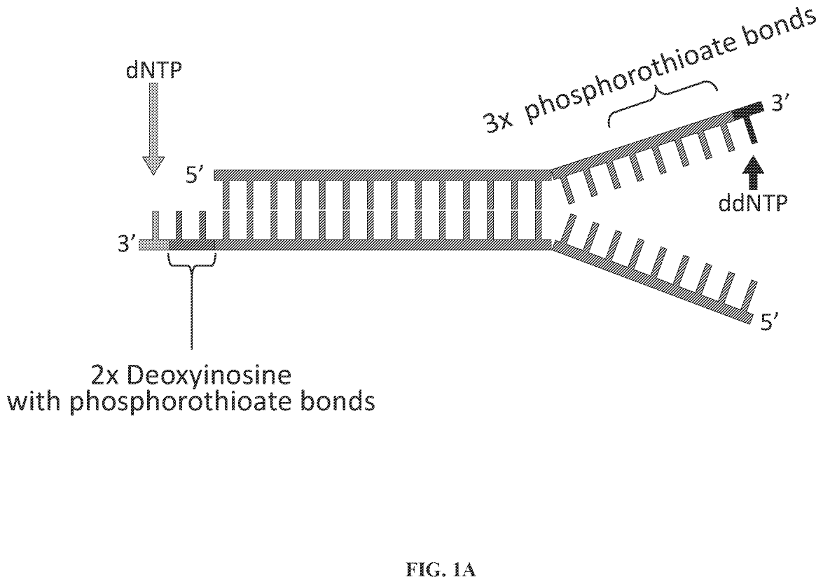

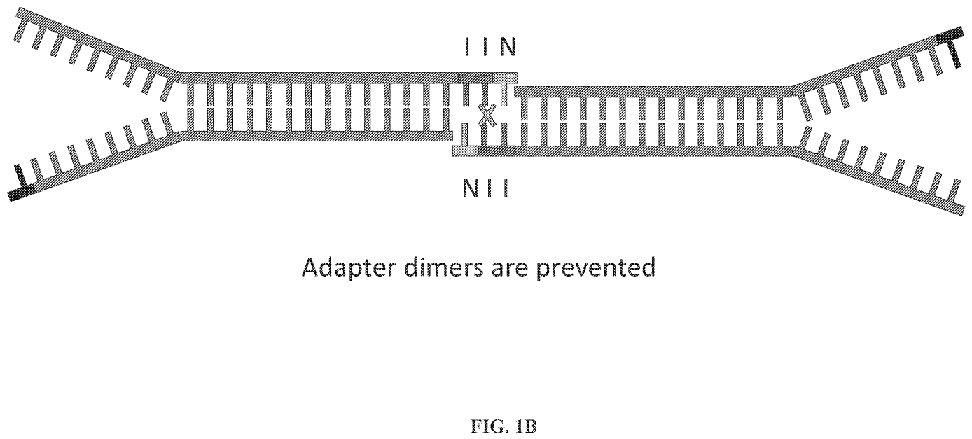

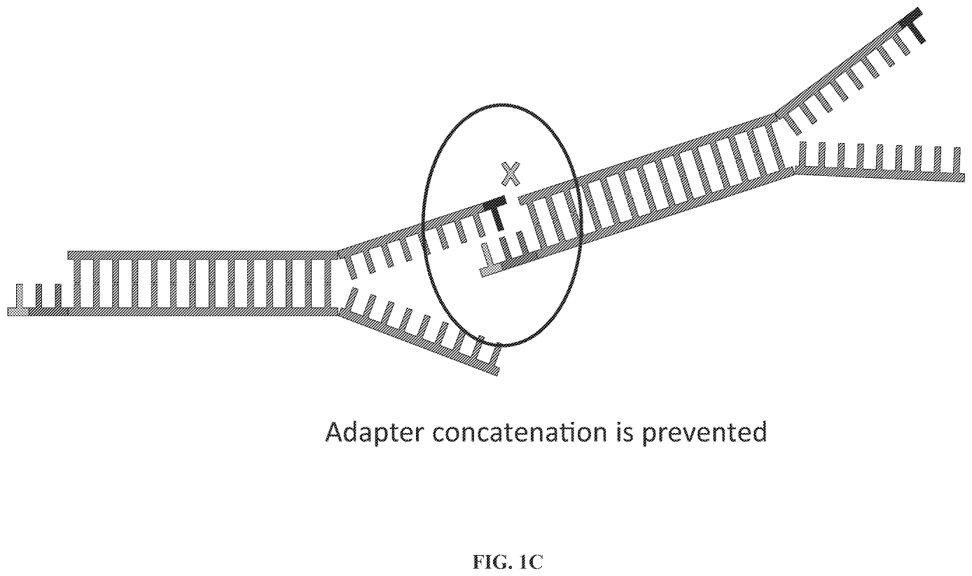

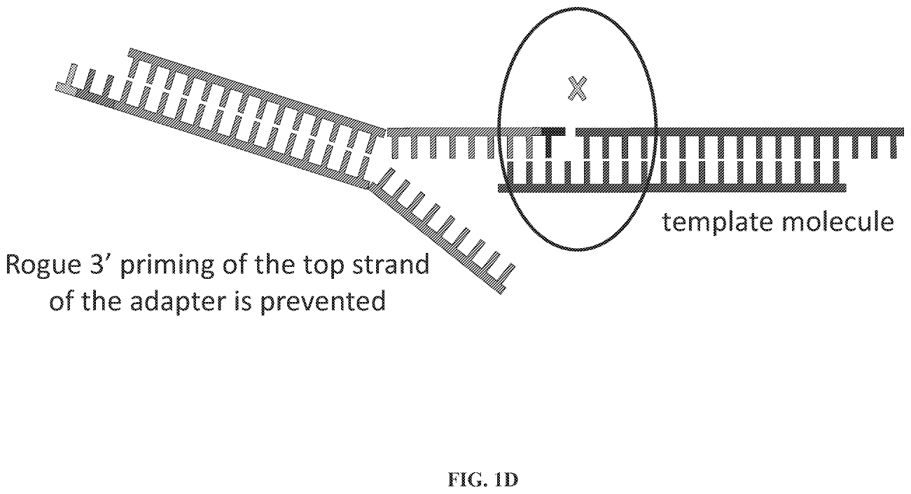

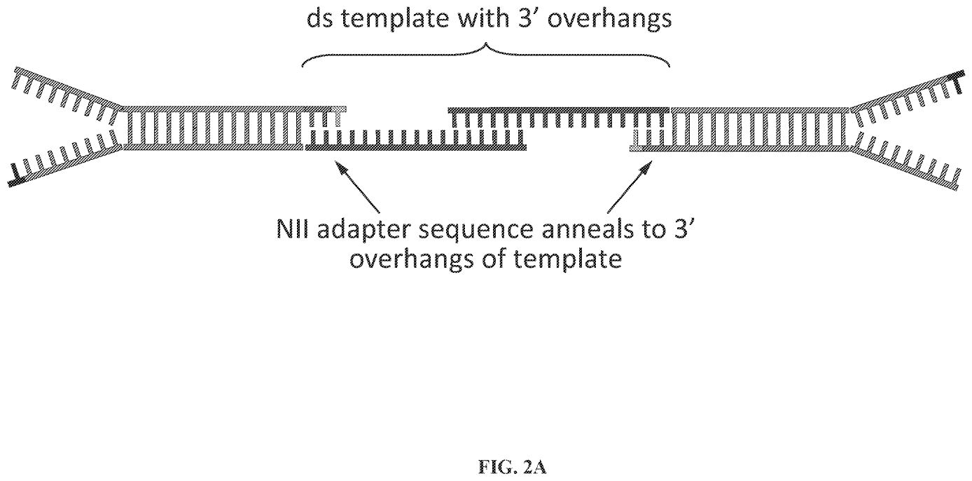

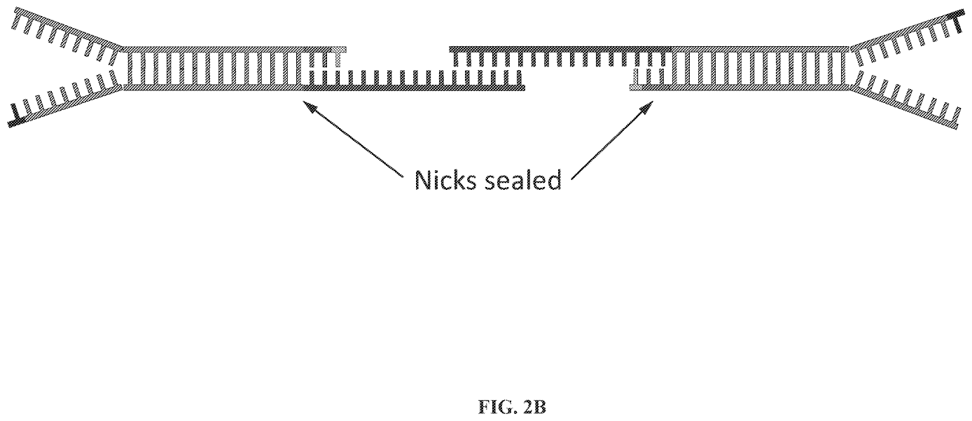

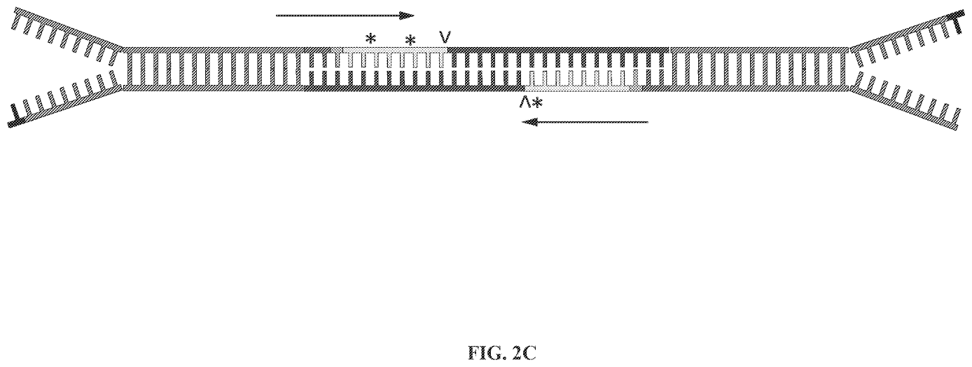

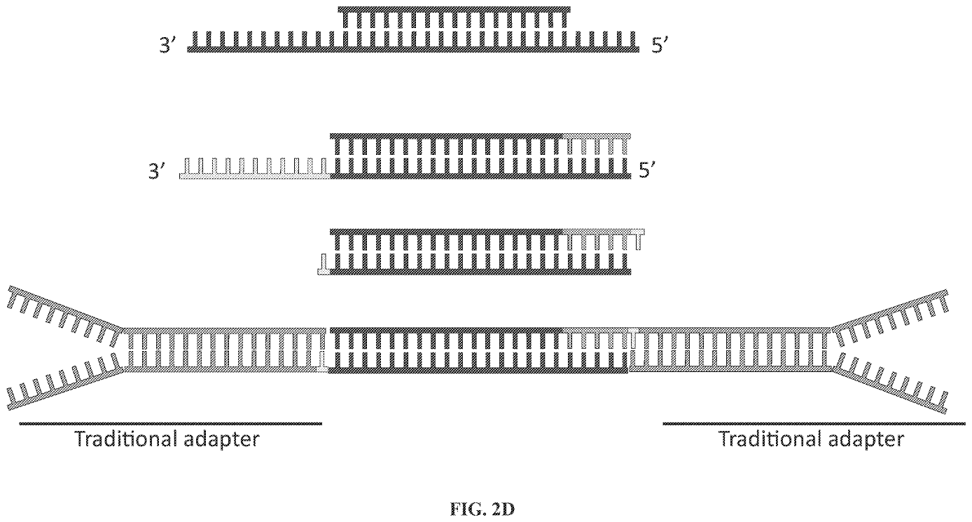

FIGS. 1A-1D illustrate an exemplary oligonucleotide (e.g., 3′ end capture adapter) of the present disclosure. FIG. 1A shows the adapter, highlighting exemplary features. The first strand (top, with 5′ to 3′ direction shown left to right) includes, from 5′ to 3′, a first portion and a second portion, with the second portion ending in 3 nucleotides joined by phosphorothioate bonds followed by a 3′ terminal dideoxynucleotide (ddNTP). The second strand (bottom, with 5′ to 3′ direction shown right to left) includes, from 5′ to 3′, a portion (e.g., third portion of the present disclosure) that does not hybridize with (or is not complementary to) the second portion of the first strand, a first portion that hybridizes with (or is at least partially complementary to) the first portion of the first strand, and a second portion with a 3′ overhang having 2 deoxyinosine nucleotides (dITPs) joined by phosphorothioate bonds followed by a 3′ terminal deoxynucleotide (dNTP). FIG. 1B illustrates that formation of adapter dimers is prevented by inclusion of dITPs in the 3′ overhang, since inosine-inosine basepairing is highly unstable (I: dITP; N: dNTP). FIG. 1C illustrates that adapter concatenation is prevented by the inclusion of the 3′ terminal ddNTP in the first strand, which prevents elongation/ligation from this end. FIG. 1D illustrates that rogue 3′ priming is also prevented by the inclusion of the 3′ terminal ddNTP in the first strand, which prevents priming of template molecules from this end.

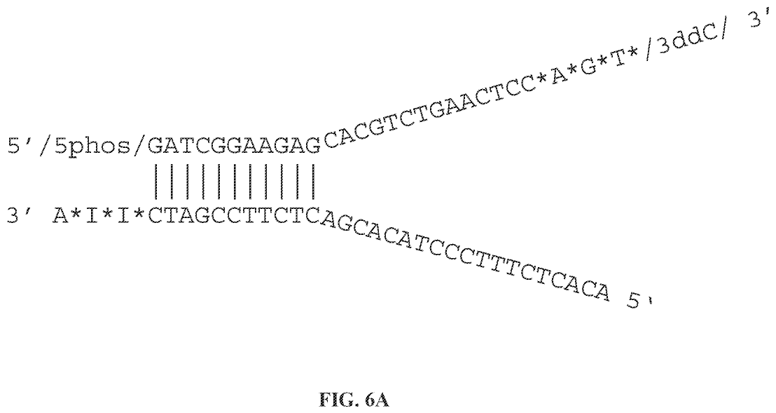

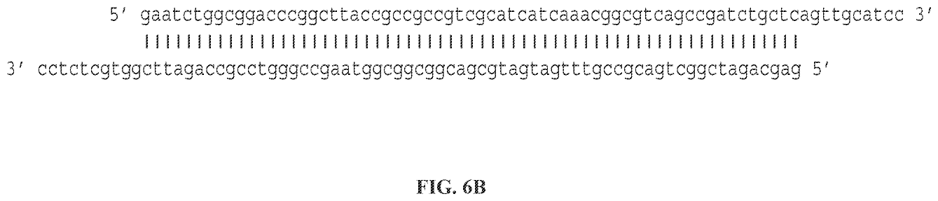

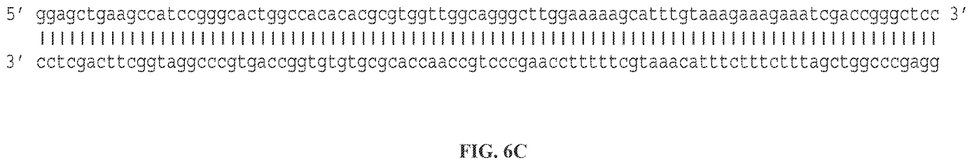

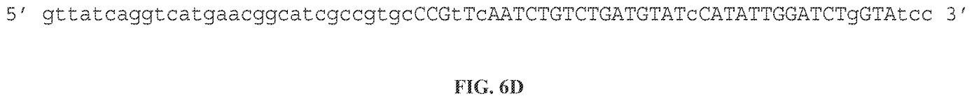

FIG. 6A provides the sequences of an exemplary oligonucleotide (e.g., 3′ end capture adapter) of the present disclosure. In some embodiments, the first strand comprises the sequence of SEQ ID NO:1. In some embodiments, the second strand comprises the sequence of SEQ ID NO: 2.

In some embodiments, the oligonucleotides are at least partially double-stranded. For example, in some embodiments, the first portion of the second strand is hybridized with the first portion of the first strand. In some embodiments, the first portion of the second strand forms a duplex with the first portion of the first strand. It is contemplated that each strand of the oligonucleotide can be manufactured separately, and the two strands annealed before, immediately prior to, or during use (e.g., contacting with template nucleic acids such as ssDNA and/or dsDNA with 3′ overhang(s)).

In some embodiments, the first portion of the first strand and/or the first portion of the second strand independently comprise 10 or more nucleotides. In some embodiments, the first portion of the first strand and/or the first portion of the second strand comprise 5 or more, 10 or more, 15 or more, or 20 or more nucleotides. In some embodiments, the first portion of the first strand and the first portion of the second strand both comprise 10 or more nucleotides. In some embodiments, the first portion of the first strand and/or the first portion of the second strand comprise less than 50, less than 40, less than 30, or less than 20 nucleotides. In some embodiments, the first portion of the first strand and/or the first portion of the second strand comprise 10-50 nucleotides, 10-40 nucleotides, 10-30 nucleotides, or 10-20 nucleotides. In some embodiments, the first portion of the first strand and the first portion of the second strand comprise the same number of nucleotides.

In some embodiments, some or all of the nucleotides of the first and/or second strands are deoxynucleotide triphosphate (dNTP) nucleotides. For example, in some embodiments, the one or more nucleotides of the first portions of the first and second strands are deoxynucleotide triphosphate (dNTP) nucleotides. In some embodiments, some or all of the first and/or second strands comprise DNA, RNA, cDNA, or modified nucleotides.

In some embodiments, the oligonucleotide adapter(s) of the present disclosure are provided in liquid form, e.g., in an aqueous solution or buffer compatible with nucleic acids (such as Tris EDTA or “TE” buffer). In some embodiments, the oligonucleotide adapter(s) of the present disclosure are provided in a solid form, e.g., in lyophilized form.

In some embodiments, the first and second strands of the oligonucleotide adapter(s) of the present disclosure are supplied separately, e.g., in separate solutions or vials. In some embodiments, the first and second strands of the oligonucleotide adapter(s) of the present disclosure are supplied together. In some embodiments, the first and second strands of the oligonucleotide adapter(s) of the present disclosure are supplied in partially double-stranded form, e.g., as shown in FIGS. 1A & 6A.

### Kits or Articles of Manufacture

Certain aspects of the present disclosure relate to kits or articles of manufacture comprising any of the oligonucleotides, e.g., oligonucleotide adapters, of the present disclosure. In some embodiments, the kits or articles of manufacture comprise four different oligonucleotide adapters of the present disclosure, each having a different 3′ terminal deoxynucleotide of the second strand (e.g., A, C, T, and G).

In some embodiments, the kits or articles of manufacture comprise: (i) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dATP; (ii) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dCTP; (iii) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dGTP; and (iv) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dTTP.

In some embodiments, the kits or articles of manufacture comprise: (i) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a third portion comprising one or more nucleotides, wherein the third portion of the second strand does not hybridize with the second portion of the first strand; a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dATP; (ii) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a third portion comprising one or more nucleotides, wherein the third portion of the second strand does not hybridize with the second portion of the first strand; a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dCTP; (iii) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a third portion comprising one or more nucleotides, wherein the third portion of the second strand does not hybridize with the second portion of the first strand; a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dGTP; and (iv) an oligonucleotide adapter comprising a first and second strand, wherein the first strand comprises (from 5′ to 3′): a first portion comprising one or more nucleotides, and a second portion comprising three or more nucleotides joined by phosphorothioate linkages followed by a 3′ terminal dideoxynucleotide (ddNTP), and wherein the second strand comprises (from 5′ to 3′): a third portion comprising one or more nucleotides, wherein the third portion of the second strand does not hybridize with the second portion of the first strand; a first portion comprising one or more nucleotides, wherein the first portion of the second strand is capable of hybridizing with the first portion of the first strand; and a second portion comprising at least two deoxyinosine nucleotides (dITPs) joined by a phosphorothioate linkage followed by a 3′ terminal dTTP.

In some embodiments, the kits or articles of manufacture further comprise a package insert or instructions for using the oligonucleotide adapter(s) of the present disclosure in accordance with any of the methods disclosed herein.

### Methods

Certain aspects of the present disclosure relate to methods for appending an oligonucleotide adapter of the present disclosure to template nucleic acid(s). Certain aspects of the present disclosure relate to methods for capturing one or more template nucleic acid(s) with an oligonucleotide adapter of the present disclosure. In some embodiments, the template nucleic acid(s) comprise single-stranded templates. In some embodiments, the template nucleic acid(s) comprise double-stranded templates with one or two 3′ overhang(s). In some embodiments, the template nucleic acid(s) comprise single-stranded templates and/or double-stranded templates with one or two 3′ overhang(s). In some embodiments, the template nucleic acid(s) comprise ssDNA fragment(s) and/or dsDNA fragments with one or two 3′ overhang(s). In some embodiments, the methods comprise: contacting an oligonucleotide adapter of the present disclosure with a plurality of template nucleic acids (e.g., ssDNA fragments and/or dsDNA fragments comprising 3′ overhang(s)) under conditions suitable for hybridization of the adapter with a ssDNA or dsDNA fragment of the plurality, wherein the second portion of the second strand of the adapter (e.g., comprising at least two dITPs joined by a phosphorothioate linkage followed by a 3′ terminal deoxynucleotide) hybridizes with a 3′ end of the ssDNA fragment of the plurality or a 3′ overhang of the dsDNA fragment of the plurality; incubating the hybridized adapter and ssDNA or dsDNA fragment with a DNA ligase under conditions suitable for sealing a nick between a 3′ end of the ssDNA fragment or overhang of the dsDNA fragment and the 5′ end of the first portion of the first strand of the adapter; and incubating the hybridized adapter and fragment with a DNA polymerase and nucleotides under conditions suitable for the polymerase to catalyze strand extension from the 3′ terminal deoxynucleotide of the second strand of the adapter using the ssDNA fragment or overhang of the dsDNA fragment as a template, thereby capturing the fragment or appending the adapter onto the fragment. As discussed herein, oligonucleotide adapters of the present disclosure and 3′ end capture oligonucleotides/adapters of the present disclosure may be referred to interchangeably and are meant to be contrasted with “traditional” adapters such as standard Y adapters that are typically appended to blunt-ended double-stranded nucleic acids or templates and/or double-stranded nucleic acids or templates with 5′ overhang(s), as opposed to the single-stranded nucleic acids or templates and/or double-stranded nucleic acids or templates with 3′ overhang(s) targeting by the oligonucleotide adapters/3′ end capture oligonucleotides/adapters of the present disclosure.

In some embodiments, the adapter comprises a first strand comprising, from 5′ to 3′: a first portion comprising one or more nucleotides, and a second portion comprising three or more consecutive nucleotides joined by phosphorothioate linkages followed by a 3′ terminal dideoxynucleotide; and a second strand comprising, from 5′ to 3′: a first portion comprising one or more nucleotides, and a second portion comprising at least two deoxyinosine nucleotides (dITPs) joined by a phosphorothioate linkage followed by a 3′ terminal deoxynucleotide, wherein the first portion of the second strand of the adapter hybridizes with (or is at least partially complementary to) the first portion of the first strand of the adapter.

In some embodiments, the adapter comprises a first strand comprising, from 5′ to 3′: a first portion comprising one or more nucleotides, and a second portion comprising three or more consecutive nucleotides joined by phosphorothioate linkages followed by a 3′ terminal dideoxynucleotide; and a second strand comprising, from 5′ to 3′: a third portion comprising one or more nucleotides, wherein the third portion of the second strand does not hybridize with the second portion of the first strand; a first portion comprising one or more nucleotides, and a second portion comprising at least two deoxyinosine nucleotides (dITPs) joined by a phosphorothioate linkage followed by a 3′ terminal deoxynucleotide, wherein the first portion of the second strand of the adapter hybridizes with (or is at least partially complementary to) the first portion of the first strand of the adapter.

In some embodiments, the template nucleic acids are contacted with a plurality of oligonucleotide adapters of the present disclosure comprising four different oligonucleotide adapters, each having a different 3′ terminal deoxynucleotide of the second strand (e.g., A, C, T, and G). In some embodiments, the template nucleic acids are contacted with a plurality of oligonucleotide adapters of the present disclosure comprising an oligonucleotide adapter having a second strand with a 3′ terminal dATP, an oligonucleotide adapter having a second strand with a 3′ terminal dCTP, an oligonucleotide adapter having a second strand with a 3′ terminal dGTP, and an oligonucleotide adapter having a second strand with a 3′ terminal dTTP.

In some embodiments, the hybridized adapter and ssDNA or dsDNA fragment are incubated with a DNA ligase under conditions suitable for ligating (e.g., sealing a nick between) the 3′ end of the ssDNA fragment or overhang of the dsDNA fragment and the 5′ end of the first portion of the first strand of the adapter. In some embodiments, the hybridized adapter and ssDNA or dsDNA fragment are incubated with a DNA ligase and a polynucleotide kinase (PNK) under conditions suitable for ligating (e.g., sealing a nick between) the 3′ end of the ssDNA fragment or overhang of the dsDNA fragment and the 5′ end of the first portion of the first strand of the adapter. For example, the 5′ end of the adapter may or may not be synthesized with a 5′ phosphate, but the PNK can ensure the presence of 5′ phosphates on the template nucleic acid and that there are no 3′ phosphates on the template nucleic acid.

In some embodiments (e.g., when the template to be captured is a double-stranded nucleic acid with one or two 3′ overhang(s)), the methods further comprise, after strand extension, incubating the hybridized adapter and fragment with a DNA ligase under conditions suitable for ligating (e.g., sealing a nick between) the strand synthesized by primer extension and a 5′ end of the double-stranded template. In some embodiments (e.g., when the template to be captured is a double-stranded nucleic acid with one or two 3′ overhang(s)), the methods further comprise, after strand extension, incubating the hybridized adapter and fragment with a DNA ligase and a PNK under conditions suitable for ligating (e.g., sealing a nick between) the strand synthesized by primer extension and a 5′ end of the double-stranded template.

Suitable DNA ligases are known in the art. In some embodiments, the DNA ligase is a T3, T4, or Taq DNA ligase. As would be recognized by a person having ordinary skill in the art, suitable conditions for hybridization of the inosine adapter to the template nucleic acid may vary, e.g., may require increased concentration of salt. Some DNA ligases (T4 for example) are not very tolerant of salt, while others like T3 are. Selection of a suitable DNA ligase depending upon reaction conditions is within ordinary skill in the art.

Suitable PNKs are known in the art. In some embodiments, the PNK is a T4 PNK. In some embodiments, the PNK is a T7 PNK or mammalian PNK. Selection of a suitable PNK depending upon reaction conditions is within ordinary skill in the art.

In some embodiments, the hybridized adapter and template nucleic acid(s) are incubated with a DNA polymerase and nucleotides under conditions suitable for the polymerase to catalyze strand extension from the 3′ terminal deoxynucleotide of the second strand of the adapter based on the template. In some embodiments, the nucleotides comprise dNTPs, e.g., dATP, dCTP, dGTP, and dTTP. In some embodiments, the nucleotides comprise one or more modified nucleotides, including without limitation 8-oxoguanine, 5-methylcytosine (5mC), or inosine. For example, 8-oxoguanine can base pair with adenine during PCR, resulting in G→T mutations when mapped. As a result, regions with high rates of G→T mutations on one strand but not the other indicate 3′ overhangs that were filled in by strand extension, thereby allowing the 3′ overhang sequences to be mapped. 5mC could be used for methyl-sequencing approaches. In some embodiments, the DNA polymerase is capable of catalyzing strand extension using the one or more modified nucleotides.

In some embodiments, the DNA polymerase lacks strand displacement activity, e.g., under the conditions used for strand extension. DNA polymerases lacking strand displacement activity are known in the art and include, without limitation, T4 DNA polymerase. In some embodiments, the DNA polymerase lacks 5′ to 3′ exonuclease activity and/or 3′ to 5′ exonuclease activity. In some embodiments, the DNA polymerase lacks 5′ to 3′ exonuclease activity and 3′ to 5′ exonuclease activity. In some embodiments, the DNA polymerase is active at temperatures below, e.g., 60° C. (e.g., such that the DNA polymerase is active under temperatures/conditions that do not promote strand denaturation).

In some embodiments, the plurality of nucleic acids contacted by the oligonucleotide adapter(s) (e.g., 3′ end capture adapters) of the present disclosure further comprises blunt-ended, double-stranded nucleic acids (e.g., dsDNA fragments) and/or double-stranded nucleic acids (e.g., dsDNA fragments) with one or two 5′ overhang(s). For example, in some embodiments, the plurality of nucleic acids contacted by the oligonucleotide adapter(s) of the present disclosure comprises: (i) single-stranded nucleic acids and/or double-stranded nucleic acids with one or two 3′ overhang(s); and (ii) blunt-ended, double-stranded nucleic acids (e.g., dsDNA fragments) and/or double-stranded nucleic acids (e.g., dsDNA fragments) with one or two 5′ overhang(s). In some embodiments, the plurality of nucleic acids contacted by the oligonucleotide adapter(s) of the present disclosure comprises single-stranded nucleic acids (e.g., ssDNA fragments), double-stranded nucleic acids (e.g., dsDNA fragments) with one or two 3′ overhang(s), blunt-ended, double-stranded nucleic acids (e.g., dsDNA fragments), and double-stranded nucleic acids (e.g., dsDNA fragments) with one or two 5′ overhang(s). In some embodiments, oligonucleotide adapter(s) (e.g., 3′ end capture adapters) of the present disclosure are contacted with a plurality of ssDNA fragments and/or dsDNA fragments comprising 3′ overhang(s) in the presence of blunt-ended, double-stranded nucleic acids (e.g., dsDNA fragments) and/or double-stranded nucleic acids (e.g., dsDNA fragments) with one or two 5′ overhang(s). Advantageously, the methods of the present disclosure can be used to append 3′ end capture adapters onto single-stranded nucleic acids (e.g., ssDNA fragments) and/or double-stranded nucleic acids (e.g., dsDNA fragments) with one or two 3′ overhang(s) prior to standard library prep methods, e.g., for capturing blunt-ended, double-stranded nucleic acids (e.g., dsDNA fragments) and/or double-stranded nucleic acids (e.g., dsDNA fragments) with one or two 5′ overhang(s).

In some embodiments, the methods of the present disclosure further comprise, e.g., after appending oligonucleotide adapter(s) onto ssDNA fragment(s) and/or 3′ overhang(s) of dsDNA fragment(s) as described above (e.g., after strand extension and optional ligation/nick sealing for double-stranded templates with 3′ overhang(s)), subjecting nucleic acids to one or more steps of traditional library prep methods, including without limitation any or all of cleanup, end-repair, 3′ adenylation, and traditional adapter ligation. In some embodiments, the nucleic acids further comprise blunt-ended, double-stranded nucleic acids (e.g., dsDNA fragments) and/or double-stranded nucleic acids (e.g., dsDNA fragments) with one or two 5′ overhang(s), resulting in ligation of traditional adapters (e.g., Y adapters) to blunt ends and/or 5′ overhangs after the traditional library prep methods (e.g., after 3′ end capture adapters of the present disclosure have already been appended to single-stranded nucleic acids and/or double-stranded nucleic acids with 3′ overhang(s) using the methods disclosed herein). In some embodiments, end-repair, 3′ adenylation, and/or traditional adapter ligation are performed in the presence of nucleic acids to which oligonucleotide adapter(s) (e.g., 3′ end capture adapter(s)) of the present disclosure) have been appended. Advantageously, this allows the products of the methods described herein to be processed by traditional library prep methods, thus enabling ssDNA and/or dsDNA with 3′ overhangs to be analyzed along with blunt-ended dsDNA and/or dsDNA with 5′ overhangs.

Traditional library prep methods are known in the art and can include, for example, the steps of sample cleanup, end-repair, 3′ adenylation, and/or adapter ligation (e.g., using traditional adapters suitable for ligation onto blunt-ended double-stranded nucleic acids and/or double-stranded nucleic acids with 5′ overhang(s)). Cleanup as used herein can refer to purification of nucleic acids of interest (e.g., for sequencing) away from one or more contaminants, including without limitation free adapter, adapter dimers, primers, unincorporated nucleotides, other reaction components, salts, proteins, surfactants, and non-nucleotide contaminants from a sample. Cleanup can be performed once or multiple times during library prep, e.g., during or after isolation of nucleic acids from a sample, prior to adapter ligation, after adapter ligation, prior to PCR, after PCR, and/or prior to sequencing. Methods and products for cleanup are known in the art; see, e.g., AMPure XP Reagent (Beckman Coulter). End-repair (also known as end-polishing) as used herein can refer to generation of blunt ends, e.g., by filling in or degrading overhangs, and/or addition of 5′ phosphates. Methods and products for end-repair (e.g., DNA polymerase and PNK) are known in the art; see, e.g., NEBNext® Ultra™ II (NEB). 3′ adenylation, also known as A-tailing or dA-tailing, as used herein can refer to adding or retaining a 3′ terminal adenine onto a nucleic acid. Methods and products for 3′ adenylation (e.g., Taq DNA polymerase) are known in the art; see, e.g., NEBNext® Ultra™ II (NEB).

Adapter ligation (e.g., using traditional adapters) can refer to ligating an adapter onto a blunt-ended double-stranded nucleic acid, double-stranded nucleic acid with 5′ overhang(s), and/or 3′ overhang(s) (e.g., A-tailed single bp 3′ overhangs with a 3′ terminal adenine as discussed supra). Traditional adapters can include, for example, binding sites for index primers, barcoding sequences, etc. Exemplary traditional adapters are known in the art; see, e.g., NEBNext® Ultra™ II Y adapters (NEB) and xGen™ Stubby Adapters (IDT).

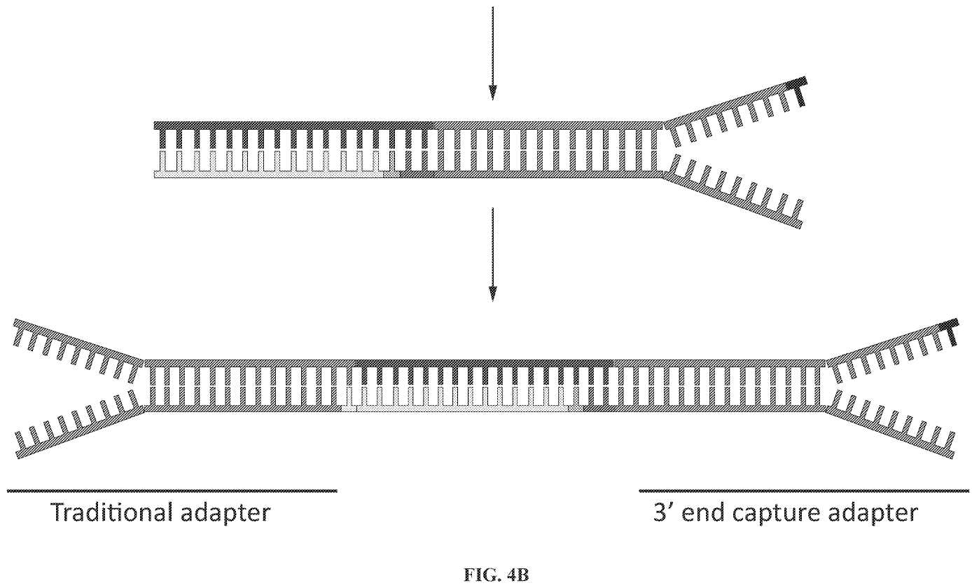

In some embodiments, the methods of the present disclosure comprise appending an oligonucleotide adapter (e.g., 3′ end capture adapter) of the present disclosure to one end of a single-stranded nucleic acid (e.g., ssDNA) as described supra, then appending a traditional adapter to the other end, e.g., using traditional library prep methods, as illustrated in FIGS. 4A & 4B.

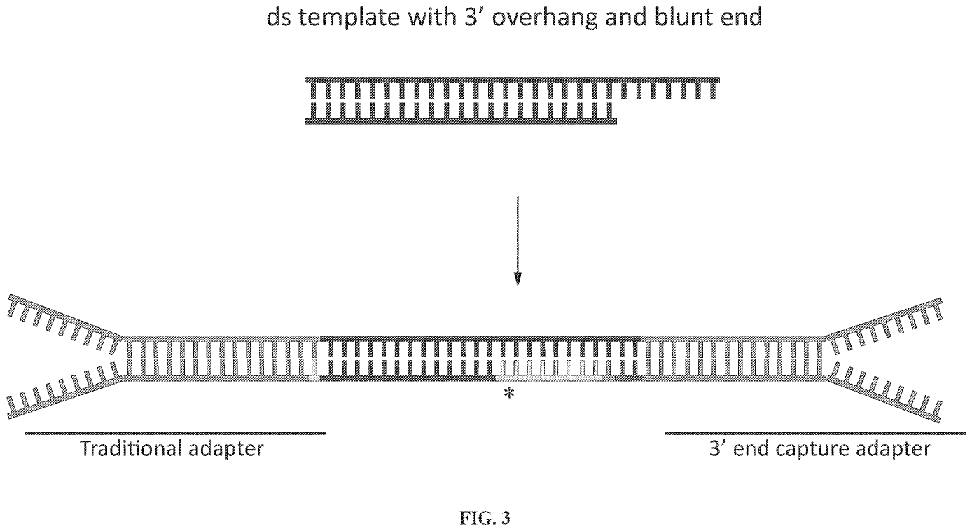

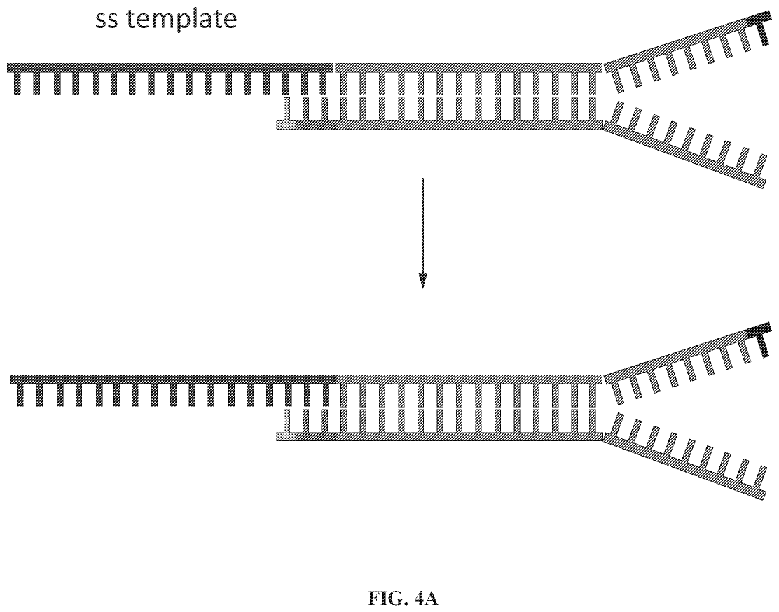

In some embodiments, the methods of the present disclosure comprise appending an oligonucleotide adapter (e.g., 3′ end capture adapter) of the present disclosure to one end (i.e., having a 3′ overhang) of a double-stranded nucleic acid (e.g., dsDNA) as described supra, then appending a traditional adapter to the other end, e.g., using traditional library prep methods, as illustrated in FIG. 3.

In some embodiments, the methods of the present disclosure further comprise, e.g., after appending oligonucleotide adapter(s) onto ssDNA fragment(s) and/or 3′ overhang(s) of dsDNA fragment(s) as described above, isolating nucleic acids with appended adapter(s) (e.g., products of strand extension) from free oligonucleotide adapters (e.g., excess adapters not appended to a ssDNA or dsDNA fragment). In some embodiments, the methods of the present disclosure further comprise, e.g., after appending oligonucleotide adapter(s) onto ssDNA fragment(s) and/or 3′ overhang(s) of dsDNA fragment(s) as described above, removing free oligonucleotide adapters (e.g., excess adapters not appended to a ssDNA or dsDNA fragment). In some embodiments, these steps separate free adapters from adapter-appended former ssDNA fragments, dsDNA fragments with former 3′ overhang(s), blunt-ended dsDNA fragments, and/or dsDNA fragments with 5′ overhang(s).

In some embodiments, the methods of the present disclosure further comprise, e.g., after appending oligonucleotide adapter(s) onto ssDNA fragment(s) and/or 3′ overhang(s) of dsDNA fragment(s) as described above, subjecting nucleic acids to PCR amplification.

In some embodiments, the methods of the present disclosure further comprise, e.g., after appending oligonucleotide adapter(s) onto ssDNA fragment(s) and/or 3′ overhang(s) of dsDNA fragment(s) as described above, subjecting nucleic acids to sequencing, e.g., NGS sequencing or methyl sequencing.

In some embodiments, e.g., when strand extension is performed with modified nucleotide(s) such as 8-oxoguanine, the methods further comprise subjecting nucleic acids to PCR amplification and subsequent sequencing (e.g., after appending oligonucleotide adapter(s) onto ssDNA fragment(s) and/or 3′ overhang(s) of dsDNA fragment(s) as described above). In some embodiments, the sequencing comprises identifying a guanine→thymine mutation corresponding to a position of an adenine nucleotide base-paired with an 8-oxoguanine during strand extension, e.g., wherein the G→T mutation is on one strand but not the other, indicating site(s) of 8-oxoguanine indicative of the presence of 3′ overhang sequence.

Various sequencing methods are known in the art, including without limitation next-generation sequencing (NGS). Next-generation sequencing generally includes any sequencing method that determines the nucleotide sequence of either individual nucleic acid molecules or clonally expanded proxies for individual nucleic acid molecules in a highly parallel fashion (e.g., greater than 105 molecules are sequenced simultaneously). In one embodiment, the relative abundance of the nucleic acid species in the library can be estimated by counting the relative number of occurrences of their cognate sequences in the data generated by the sequencing experiment.

NGS methods are known in the art, and are described, e.g., in Metzker, M. (2010) Nature Biotechnology Reviews 11:31-46. Platforms for next-generation sequencing include, e.g., Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, and Pacific Biosciences' PacBio RS system. In one embodiment, the next-generation sequencing allows for the determination of the nucleotide sequence of an individual nucleic acid molecule (e.g., Helicos BioSciences' HeliScope Gene Sequencing system, and Pacific Biosciences' PacBio RS system). In other embodiments, the sequencing method determines the nucleotide sequence of clonally expanded proxies for individual nucleic acid molecules (e.g., the Solexa sequencer, Illumina Inc., San Diego, Calif; 454 Life Sciences (Branford, Conn.), and Ion Torrent), e.g., massively parallel short-read sequencing (e.g., the Solexa sequencer, Illumina Inc., San Diego, Calif.), which generates more bases of sequence per sequencing unit than other sequencing methods that generate fewer but longer reads.

NGS technologies can include one or more of steps, e.g., template preparation, sequencing and imaging, and data analysis. Methods for template preparation can include steps such as randomly breaking nucleic acids (e.g., genomic DNA) into smaller sizes and generating sequencing templates (e.g., fragment templates or mate-pair templates). The spatially separated templates can be attached or immobilized to a solid surface or support, allowing massive amounts of sequencing reactions to be performed simultaneously. Types of templates that can be used for NGS reactions include, e.g., clonally amplified templates originating from single DNA molecules, and single DNA molecule templates. Exemplary sequencing and imaging steps for NGS include, e.g., cyclic reversible termination (CRT), sequencing by ligation (SBL), single-molecule addition (pyrosequencing), and real-time sequencing. After NGS reads have been generated, they can be aligned to a known reference sequence or assembled de novo. For example, identifying genetic variations such as single-nucleotide polymorphism and structural variants in a sample (e.g., a tumor sample) can be accomplished by aligning NGS reads to a reference sequence (e.g., a wild type sequence). Methods of sequence alignment for NGS are described e.g., in Trapnell C. and Salzberg S. L. Nature Biotech., 2009, 27:455-457. Examples of de novo assemblies are described, e.g., in Warren R. et al., Bioinformatics, 2007, 23:500-501; Butler J. et al., Genome Res., 2008, 18:810-820; and Zerbino D. R. and Birney E., Genome Res., 2008, 18:821-829. Sequence alignment or assembly can be performed using read data from one or more NGS platforms, e.g., mixing Roche/454 and Illumina/Solexa read data. In some embodiments, NGS is performed according to the methods described in, e.g., Frampton, G. M. et al. (2013) Nat. Biotech. 31:1023-1031; and/or Montesion, M., et al., Cancer Discovery (2021) 11 (2): 282-92.

In some embodiments, sequencing includes paired-end sequencing or unpaired sequencing. Generally, paired-end sequencing methodologies are described, e.g., in WO2007/010252, WO2007/091077, and WO03/74734. This approach utilizes pairwise sequencing of a double-stranded polynucleotide template, which results in the sequential determination of nucleotide sequences in two distinct and separate regions of the polynucleotide template. The paired-end methodology makes it possible to obtain two linked or paired reads of sequence information from each double-stranded template on a clustered array, rather than just a single sequencing read as can be obtained with other methods. Paired end sequencing technology can make special use of clustered arrays, generally formed by solid-phase amplification, for example as set forth in WO03/74734. Target polynucleotide duplexes, fitted with adapters, are immobilized to a solid support at the 5′ ends of each strand of each duplex, for example, via bridge amplification as described above, forming dense clusters of double stranded DNA. Because both strands are immobilized at their 5′ ends, sequencing primers are then hybridized to the free 3′ end and sequencing by synthesis is performed. Adapter sequences can be inserted in between target sequences to allow for up to four reads from each duplex, as described in WO2007/091077. In a further adaptation of this methodology, specific strands can be cleaved in a controlled fashion as set forth in WO2007/010252. As a result, the timing of the sequencing read for each strand can be controlled, permitting sequential determination of the nucleotide sequences in two distinct and separate regions on complementary strands of the double-stranded template. See, e.g., U.S. Pat. No. 10,174,372.

In some embodiments, a hybrid capture approach is used. Further details about this and other hybrid capture processes can be found in U.S. Pat. No. 9,340,830; Frampton, G. M. et al. (2013) Nat. Biotech. 31:1023-1031; and Montesion, M., et al., Cancer Discovery (2021) 11 (2): 282-92. In some embodiments, the methods further comprise, prior to contacting the mixture of polynucleotides with the bait molecule: obtaining a sample from an individual, wherein the sample comprises tumor cells and/or tumor nucleic acids; and extracting the mixture of polynucleotides from the sample, wherein the mixture of polynucleotides is from the tumor cells and/or tumor nucleic acids. In some embodiments, the sample further comprises non-tumor cells.

In some embodiments, the methods further comprise selectively enriching for a plurality of nucleic acids or nucleic acid fragment. For example, one or more baits or probes can be used to hybridize with a genomic locus of interest or fragment thereof, e.g., comprising a cluster of two or more CpG dinucleotides or comprising a genetic variant/mutation of interest. See, e.g., Graham, B. I. et al. Twist Fast Hybridization targeted methylation sequencing: a tunable target enrichment solution for methylation detection [abstract]. In: Proceedings of the American Association for Cancer Research Annual Meeting 2021; 2021 Apr. 10-15 and May 17-21. Philadelphia (PA): AACR; Cancer Res 2021; 81 (13_Suppl): Abstract nr 2098.

Generally, methyl sequencing methods (e.g., bisulfite sequencing, EM-Seq, etc.) involve cytosine conversion followed by sequence analysis. Cytosine conversion is typically used to mark cytosines based on methylation status. For example, bisulfite treatment converts cytosine to uracil but does not alter 5mC. Subsequent analysis can reveal methylation state by identifying which base pairs were converted and which were not.

Methods for methyl sequencing are known in the art, including whole-genome methyl sequencing. Generally, these methods combine cytosine conversion with sequencing techniques. For example, in some embodiments, the methyl sequencing comprises bisulfite sequencing, whole genome bisulfite sequencing (WGBS), APOBEC-seq, methyl-CpG-binding domain (MBD) protein capture, methyl-DNA immunoprecipitation (MeDIP-seq), methylation sensitive restriction enzyme sequencing (MSRE/MRE-Seq or Methyl-Seq), enzymatic methylation sequencing, oxidative bisulfite sequencing (oxBS-Seq), reduced representative bisulfite sequencing (RRBS), or Tet-assisted bisulfite sequencing (TAB-Seq).

A commonly-used method of determining the methylation level and/or pattern of DNA requires methylation status-dependent conversion of cytosine in order to distinguish between methylated and non-methylated CpG dinucleotide sequences. For example, methylation of CpG dinucleotide sequences can be measured by employing cytosine conversion based technologies, which rely on methylation status-dependent chemical modification of CpG sequences within isolated genomic DNA, or fragments thereof, followed by DNA sequence analysis. Chemical reagents that are able to distinguish between methylated and non-methylated CpG dinucleotide sequences include hydrazine, which cleaves the nucleic acid, and bisulfite treatment. Bisulfite treatment followed by alkaline hydrolysis specifically converts non-methylated cytosine to uracil, leaving 5-methylcytosine unmodified as described by Olek A., Nucleic Acids Res. 24:5064-6, 1996 or Frommer et al., Proc. Natl. Acad. Sci. USA 89:1827-1831 (1992). The bisulfite-treated DNA can subsequently be analyzed by conventional molecular techniques, such as PCR amplification, sequencing, and detection comprising oligonucleotide hybridization. See, e.g., U.S. Pat. No. 10,174,372.

Various methodologies for cytosine conversion are known in the art. In some embodiments, a plurality of nucleic acids or nucleic acid fragments of the present disclosure has undergone cytosine conversion by bisulfite treatment, TET-assisted bisulfite treatment, TET-assisted pyridine borane treatment, oxidative bisulfite treatment, or APOBEC treatment, e.g., prior to detection.

As such, in some embodiments, the methods of the present disclosure comprise treating a plurality of nucleic acids or nucleic acid fragments of the present disclosure with bisulfite.

Bisulfite sequencing is a commonly used method in the art for generating methylation data at single-base resolution. Bisulfite conversion or treatment refers to a biochemical process for converting unmethylated cytosine residue to uracil or thymine residues (e.g., deamination to uracil, followed by amplification as thymine during PCR), whereby methylated cytosine residues (e.g., 5-methylcytosine, 5mC; or 5-hydroxymethylcytosine, 5hmC) are preserved. Reagents to convert cytosine to uracil are known to those of skill in the art and include bisulfite reagents such as sodium bisulfite, potassium bisulfite, ammonium bisulfite, magnesium bisulfite, sodium metabisulfite, potassium metabisulfite, ammonium metabisulfite, magnesium metabisulfite and the like.

In some embodiments, the methods of the present disclosure comprise treating a plurality of nucleic acids or nucleic acid fragments of the present disclosure with enzymatic digestion and bisulfite treatment. The principle of the method is that the fragmentation of DNA is not achieved by ultrasound but achieved by combined enzymatic digestion by multiple endonucleases (MseI, Tsp 509I, NlaIII and Hpy CH₄V), wherein the restriction enzyme cutting sites of MseI, Tsp509I, NlaIII and Hpy CH₄V are TTAA, AATT, CATG and TGCA, respectively. See, e.g., Smiraglia D J, et al. Oncogene 2002; 21:5414-5426. This is followed by bisulfite treatment, e.g., as described herein.

Enzymatic methods for cytosine conversion are also known, e.g., enzymatic methyl sequencing. Such approaches can be advantageous because they employ enzymes instead of bisulfite, which can damage and fragment DNA, leading to DNA loss and potentially biased sequencing. For example, TET2 (the Ten-eleven translocation (Tet) family 2 methylcytosine dioxygenase) and T4-BGT (T4 phage beta-glucosyltransferase) can be used to convert 5mC and 5hmC into products that cannot be deaminated by APOBEC3A (apolipoprotein B mRNA editing enzyme, catalytic polypeptide-like 3A), then APOBEC3A is used to deaminate unmodified cytosines by converting them into uracils. See, e.g., Vaisvila, R. et al. (2021) Genome Res. 31:1-10.

In some embodiments, the methods of the present disclosure comprise treating a plurality of nucleic acids or nucleic acid fragments of the present disclosure with TET-assisted bisulfite (e.g., TAB-seq). In the TAB-seq approach, beta-glucosyltransferase (BGT) is used to convert 5hmC into β-glucosyl-5-hydroxymethylcytosine (5gmC), and a Tet enzyme (e.g., mTet1) is used to oxidize 5mC into 5-carboxylcytosine (5caC). Subsequently, nucleic acids can be treated with bisulfite. See, e.g., Yu, M. et al. (2018) Methods Mol. Biol. 1708:645-663.

In some embodiments, the methods of the present disclosure comprise treating a plurality of nucleic acids or nucleic acid fragments of the present disclosure with TET-assisted pyridine borane (e.g., TAPS). In the TAPS approach, a TET methylcytosine dioxygenase is used to oxidize 5mC and 5hmC into 5caC, then 5caC is reduced into dihydrouracil (DHU) via pyridine borane. DHU is converted to thymine during subsequent PCR. See, e.g., Liu, Y. et al. (2019) Nat. Biotechnol. 37:424-429.

In some embodiments, the methods of the present disclosure comprise treating a plurality of nucleic acids or nucleic acid fragments of the present disclosure with oxidative bisulfite (e.g., oxBS). In the oxBS approach, 5hmC is oxidized into 5-formylcytosine (5fC), which can be converted to uracil under bisulfite. Sequencing results from bisulfite vs. oxidative bisulfite treatment can then be used to infer 5hmC levels from 5mC. See, e.g., Booth, M. J. et al. (2013) Nat. Protocols 8:1841-1851. This approach can be scaled on a genome-wide level in oxBS-seq; see, e.g., Kirschner, K. et al. (2018) Methods Mol. Biol. 1708:665-678.

In some embodiments, the methods of the present disclosure comprise treating a plurality of nucleic acids or nucleic acid fragments of the present disclosure with APOBEC. Enzymatic reagents to convert cytosine to uracil, i.e. cytosine deaminases, include those of the APOBEC family, such as APOBEC-seq or APOBEC3A. The APOBEC family members are cytidine deaminases that convert cytosine to uracil while maintaining 5-methyl cytosine, i.e. without altering 5-methyl cytosine. Such enzymes are described in US2013/0244237 and WO2018165366 and are commercially available (see, e.g., the NEBNext® Enzymatic Methyl-seq Kit, New England Biolabs). Non-limiting examples of APOBEC family proteins include APOBEC1, APOBEC2, APOBEC3A, APOBEC3B, APOBEC3C, APOBEC3D, APOBEC3F, APOBEC3G, APOBEC3H, APOBEC4, and Activation-induced (cytidine) deaminase.

Some methyl sequencing methods rely upon library construction and adapter ligation, followed by standard bisulfite conversion and sequencing (e.g., WGBS). Alternatively, bisulfite treatment can be carried out prior to adaptor ligation (see, e.g., Miura, F. et al. (2012) Nucleic Acids Res. 40: e136). More recent techniques use other cytosine conversion methods such as enzymatic approaches in order to reduce damage to DNA caused by bisulfite, e.g., as in the commercially available NEBNext® Enzymatic Methyl-seq Kit (New England Biolabs). Steps of library amplification, quantification, and sequencing generally follow bisulfite conversion. In some embodiments, prior to WGMS, nucleic acids are extracted from a sample. In some embodiments, prior to WGMS, nucleic acids are subjected to fragmentation, repair, and adaptor ligation. As noted previously, cytosine conversion can be carried out before or after adaptor ligation. In some embodiments, DNA repair is performed after cytosine conversion. PCR amplification (generally at least two cycles) is performed after cytosine conversion to convert uracils (generated by formerly unmethylated cytosines) into thymine, and is accomplished using a polymerase that is able to read uracil (excluding polymerases with proofreading and repair activities). In some embodiments, prior to sequencing, fragments are enriched for desired length. In some embodiments, prior to sequencing, nucleic acids are enriched for methylated sequences, such as by immunoprecipitation using an antibody specific for 5mC as in the MeDIP approach (see, e.g., Pomraning, K. R. et al. (2009) Methods 47:142-150.

### Samples

In some embodiments, template nucleic acids of the present disclosure (e.g., single-stranded nucleic acids and/or double-stranded nucleic acids with one or two 3′ overhang(s)) are obtained or isolated from a sample. In some embodiments, the methods of the present disclosure further comprise isolating a plurality of nucleic acids from a sample, e.g., prior to contacting with an oligonucleotide adapter as disclosed herein. In some embodiments, the plurality of nucleic acids comprises single-stranded nucleic acids (e.g., ssDNA fragments) and/or double-stranded nucleic acids (e.g., dsDNA fragments) with one or two 3′ overhang(s). In some embodiments, the plurality of nucleic acids further comprises blunt-ended, double-stranded nucleic acids (e.g., dsDNA fragments) and/or double-stranded nucleic acids (e.g., dsDNA fragments) with one or two 5′ overhang(s).

In some embodiments, nucleic acids are obtained from a sample, e.g., comprising tumor cells and/or tumor nucleic acids. For example, the sample can comprise tumor cell(s), circulating tumor cell(s), tumor nucleic acids (e.g., tumor circulating tumor DNA, cfDNA, or cfRNA), part or all of a tumor biopsy, fluid, cells, tissue, mRNA, cDNA, DNA, RNA, cell-free DNA, and/or cell-free RNA. In some embodiments, the sample is from a tumor biopsy or tumor specimen. In some embodiments, the sample further comprises non-tumor cells and/or non-tumor nucleic acids. In some embodiments, the fluid comprises blood, serum, plasma, saliva, semen, cerebral spinal fluid, amniotic fluid, peritoneal fluid, interstitial fluid, etc. In some embodiments, the sample further comprises non-tumor cells and/or non-tumor nucleic acids.

In some embodiments, a sample comprises tissue, cells, and/or nucleic acids from a cancer and/or tissue, cells, and/or nucleic acids from normal tissue. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, serum, cerebrospinal fluid, sputum, stool, urine, or saliva.

In some embodiments, the sample comprises a fraction of tumor nucleic acids that is less than 1% of total nucleic acids, less than 0.5% of total nucleic acids, less than 0.1% of total nucleic acids, or less than 0.05% of total nucleic acids. In some embodiments, the sample comprises a fraction of tumor nucleic acids that is at least 0.01%, at least 0.05%, or at least 0.1% of total nucleic acids. In some embodiments, the sample comprises a fraction of tumor nucleic acids having an upper limit of 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, or 0.02% of total nucleic acids and an independently selected lower limit of 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% of total nucleic acids, wherein the upper limit is greater than the lower limit.

In some embodiments, the sample is or comprises biological tissue or fluid. The sample can contain compounds that are not naturally intermixed with the tissue in nature such as preservatives, anticoagulants, buffers, fixatives, nutrients, antibiotics or the like. In one embodiment, the sample is preserved as a frozen sample or as a formaldehyde- or paraformaldehyde-fixed paraffin-embedded (FFPE) tissue preparation. For example, the sample can be embedded in a matrix, e.g., an FFPE block or a frozen sample. In another embodiment, the sample is a blood or blood constituent sample. In yet another embodiment, the sample is a bone marrow aspirate sample. In another embodiment, the sample comprises cell-free DNA (cfDNA) or circulating cell-free DNA (ccfDNA), e.g., tumor cfDNA or tumor ccfDNA. Without wishing to be bound by theory, it is believed that in some embodiments, cfDNA is DNA from apoptosed or necrotic cells. Typically, cfDNA is bound by protein (e.g., histone) and protected by nucleases. CfDNA can be used as a biomarker, for example, for non-invasive prenatal testing (NIPT), organ transplant, cardiomyopathy, microbiome, and cancer. In another embodiment, the sample comprises circulating tumor DNA (ctDNA). Without wishing to be bound by theory, it is believed that in some embodiments, ctDNA is cfDNA with a genetic or epigenetic alteration (e.g., a somatic alteration or a methylation signature) that can discriminate it originating from a tumor cell versus a non-tumor cell. In another embodiment, the sample comprises circulating tumor cells (CTCs). Without wishing to be bound by theory, it is believed that in some embodiments, CTCs are cells shed from a primary or metastatic tumor into the circulation. In some embodiments, CTCs apoptose and are a source of ctDNA in the blood/lymph.

In some embodiments, a sample of the present disclosure is obtained from an individual. In some embodiments, the individual has cancer. In some embodiments, the individual is suspected of having cancer. In some embodiments, the individual is being screened for cancer, or a recurrence or remission thereof. In some embodiments, the individual is undergoing or has undergone a treatment, e.g., for cancer.

### IV. Exemplary Embodiments

The following exemplary embodiments are representative of some aspects of the invention:
Embodiment 1. An oligonucleotide, comprising:
   a) a first strand that comprises, from 5′ to 3′:
      i) a first portion comprising one or more nucleotides, and
      ii) a second portion comprising three or more consecutive nucleotides joined by phosphorothioate linkages followed by a 3′ terminal dideoxynucleotide (ddNTP); and
   b) a second strand that comprises, from 5′ to 3′:
      i) a first portion comprising one or more nucleotides, wherein the first portion of the second strand is capable of hybridizing with the first portion of the first strand; and
      ii) a second portion comprising at least two deoxyinosine nucleotides (dITPs) joined by a phosphorothioate linkage followed by a 3′ terminal deoxynucleotide;
   wherein upon hybridization of the first portion of the first strand with the first portion of the second strand, the second portion of the second strand forms a 3′ overhang.
Embodiment 2. The oligonucleotide of embodiment 1, wherein the 3′ terminal deoxynucleotide of the second strand is a deoxyadenosine trisphosphate (dATP), deoxycytidine triphosphate (dCTP), deoxyguanosine triphosphate (dGTP), or deoxythymidine triphosphate (dTTP).
Embodiment 3. The oligonucleotide of embodiment 1 or embodiment 2, wherein the one or more nucleotides of the first portions of the first and second strands are deoxynucleotide triphosphate (dNTP) nucleotides.
Embodiment 4. The oligonucleotide of any one of embodiments 1-3, wherein the first portion of the first strand and the first portion of the second strand each independently comprise 10 or more nucleotides.
Embodiment 5. The oligonucleotide of any one of embodiments 1-4, wherein the second portion of the second strand comprises at least two consecutive dITPs joined by a phosphorothioate linkage followed by the 3′ terminal deoxynucleotide.
Embodiment 6. The oligonucleotide of embodiment 5, wherein the second portion of the second strand comprises two consecutive dITPs joined by a phosphorothioate linkage followed by the 3′ terminal deoxynucleotide.
Embodiment 7. The oligonucleotide of any one of embodiments 1-6, wherein the second strand further comprises a third portion comprising one or more nucleotides, wherein the third portion is 5′ of the first portion, and wherein, upon hybridization of the first portion of the first strand with the first portion of the second strand, the third portion of the second strand does not hybridize with the second portion of the first strand.
Embodiment 8. A kit of oligonucleotides, comprising:
   a) a first oligonucleotide according to any one of embodiments 2-7, wherein the 3′ terminal deoxynucleotide of the second strand is a deoxyadenosine trisphosphate (dATP);
   b) a second oligonucleotide according to any one of embodiments 2-7, wherein the 3′ terminal deoxynucleotide of the second strand is a deoxycytidine triphosphate (dCTP);
   c) a third oligonucleotide according to any one of embodiments 2-7, wherein the 3′ terminal deoxynucleotide of the second strand is a deoxyguanosine triphosphate (dGTP); and
   d) a fourth oligonucleotide according to any one of embodiments 2-7, wherein the 3′ terminal deoxynucleotide of the second strand is a deoxythymidine triphosphate (dTTP).
Embodiment 9. A method for appending an oligonucleotide adapter to a single-stranded DNA (ssDNA) fragment or double-stranded DNA (dsDNA) fragment comprising 3′ overhang(s), the method comprising:
   a) contacting an oligonucleotide adapter with a plurality of ssDNA fragments and/or dsDNA fragments comprising 3′ overhang(s) under conditions suitable for hybridization of the adapter with a ssDNA or dsDNA fragment of the plurality, wherein the adapter comprises:
      a first strand comprising, from 5′ to 3′: a first portion comprising one or more nucleotides, and a second portion comprising three or more consecutive nucleotides joined by phosphorothioate linkages followed by a 3′ terminal dideoxynucleotide; and
      a second strand comprising, from 5′ to 3′: a first portion comprising one or more nucleotides, and a second portion comprising at least two deoxyinosine nucleotides (dITPs) joined by a phosphorothioate linkage followed by a 3′ terminal deoxynucleotide;
      wherein the first portion of the second strand of the adapter hybridizes with the first portion of the first strand of the adapter; and
         wherein the second portion of the second strand of the adapter hybridizes with a 3′ end of the ssDNA fragment of the plurality or a 3′ overhang of the dsDNA fragment of the plurality;
   b) incubating the hybridized adapter and ssDNA or dsDNA fragment with a DNA ligase under conditions suitable for sealing a nick between a 3′ end of the ssDNA fragment or overhang of the dsDNA fragment and the 5′ end of the first portion of the first strand of the adapter; and
   c) incubating the hybridized adapter and fragment with a DNA polymerase and nucleotides under conditions suitable for the polymerase to catalyze strand extension from the 3′ terminal deoxynucleotide of the second strand of the adapter using the ssDNA fragment or overhang of the dsDNA fragment as a template, wherein the DNA polymerase lacks strand displacement activity, thereby appending the adapter onto the fragment.
Embodiment 10. The method of embodiment 9, wherein b) comprises incubating the hybridized adapter and ssDNA or dsDNA fragment with the DNA ligase and a polynucleotide kinase (PNK) under conditions suitable for sealing a nick between a 3′ end of the ssDNA fragment or overhang of the dsDNA fragment and the 5′ end of the first portion of the first strand of the adapter.
Embodiment 11. The method of embodiment 9 or embodiment 10, wherein the adapter is hybridized with a dsDNA fragment comprising a 3′ overhang, and the method further comprises, after strand extension in c), incubating the hybridized adapter and fragment with a DNA ligase under conditions suitable for sealing a nick between the strand synthesized in c) and a 5′ end of the dsDNA fragment.
Embodiment 12. The method of embodiment 11, wherein the method further comprises, after strand extension in c), incubating the hybridized adapter and fragment with the DNA ligase and a PNK under conditions suitable for sealing a nick between the strand synthesized in c) and a 5′ end of the dsDNA fragment.
Embodiment 13. The method of any one of embodiments 9-12, wherein the 3′ terminal deoxynucleotide of the second strand is a deoxyadenosine trisphosphate (dATP), deoxycytidine triphosphate (dCTP), deoxyguanosine triphosphate (dGTP), or deoxythymidine triphosphate (dTTP).
Embodiment 14. The method of any one of embodiments 9-13, wherein the one or more nucleotides of the first portions of the first and second strands are deoxynucleotide triphosphate (dNTP) nucleotides.
Embodiment 15. The method of any one of embodiments 9-14, wherein the first portion of the first strand and the first portion of the second strand each comprise 10 or more nucleotides.
Embodiment 16. The method of any one of embodiments 9-15, wherein the second portion of the second strand comprises at least two consecutive dITPs joined by a phosphorothioate linkage followed by the 3′ terminal deoxynucleotide.
Embodiment 17. The method of embodiment 16, wherein the second portion of the second strand comprises two consecutive dITPs joined by a phosphorothioate linkage followed by the 3′ terminal deoxynucleotide.
Embodiment 18. The method of any one of embodiments 9-17, wherein the second strand further comprises a third portion comprising one or more nucleotides, wherein the third portion is 5′ of the first portion, and wherein, upon hybridization of the first portion of the first strand with the first portion of the second strand, the third portion of the second strand does not hybridize with the second portion of the first strand.
Embodiment 19. The method of any one of embodiments 9-18, wherein the DNA polymerase is a T4 DNA polymerase.
Embodiment 20. The method of any one of embodiments 9-19, wherein the DNA ligase is a T3 DNA ligase, T4 DNA ligase, or Taq DNA ligase.
Embodiment 21. The method of any one of embodiments 9-20, wherein the plurality of ssDNA fragments and/or dsDNA fragments comprising 3′ overhang(s) are contacted in a) with a plurality of adapters that comprises a first adapter with deoxyadenosine trisphosphate (dATP) as the 3′ terminal deoxynucleotide of the second strand, a second adapter with deoxycytidine triphosphate (dCTP) as the 3′ terminal deoxynucleotide of the second strand, a third adapter with deoxyguanosine triphosphate (dGTP) as the 3′ terminal deoxynucleotide of the second strand, and a fourth adapter with deoxythymidine triphosphate (dTTP) as the 3′ terminal deoxynucleotide of the second strand.
Embodiment 22. The method of any one of embodiments 9-21, wherein the nucleotides of c) comprise at least one modified nucleotide.
Embodiment 23. The method of embodiment 22, wherein the modified nucleotide is 8-oxoguanine, 5-methylcytosine (5mC), or inosine.
Embodiment 24. The method of any one of embodiments 9-23, further comprising, prior to a), isolating the plurality of ssDNA fragments and/or dsDNA fragments comprising 3′ overhang(s) from a sample.
Embodiment 25. The method of embodiment 24, wherein the sample comprises cell-free DNA (cfDNA), circulating cell-free DNA (ccfDNA), or circulating tumor DNA (ctDNA).
Embodiment 26. The method of embodiment 24, wherein the sample comprises fluid, cells, or tissue.
Embodiment 27. The method of embodiment 24, wherein the sample comprises tumor cells and/or tumor nucleic acids.
Embodiment 28. The method of any one of embodiments 24-27, wherein the sample further comprises blunt-ended dsDNA fragments and/or dsDNA fragments comprising 5′ overhang(s), and wherein the method further comprises, prior to a), isolating the blunt-ended dsDNA fragments and/or dsDNA fragments comprising 5′ overhang(s) with the plurality of ssDNA fragments and/or dsDNA fragments comprising 3′ overhang(s).
Embodiment 29. The method of embodiment 28, further comprising, after strand extension in c):
   (i) subjecting the blunt-ended dsDNA fragments and/or dsDNA fragments comprising 5′ overhang(s) to end repair;
   (ii) subjecting the end-repaired dsDNA fragments to 3′ adenylation; and
   (iii) subjecting the 3′ adenylated dsDNA fragments to adapter ligation.
Embodiment 30. The method of embodiment 29, wherein (i)-(iii) are performed in the presence of the product of strand extension from c).
Embodiment 31. The method of any one of embodiments 9-30, further comprising, after strand extension in c), isolating the product of strand extension from free oligonucleotide adapters.
Embodiment 32. The method of any one of embodiments 9-31, further comprising, after strand extension in c), subjecting the product of strand extension to polymerase chain reaction (PCR) amplification.
Embodiment 33. The method of any one of embodiments 9-32, further comprising, after strand extension in c), subjecting the product of strand extension or PCR amplicon(s) thereof to sequencing or methyl sequencing.
Embodiment 34. The method of embodiment 33, wherein the nucleotides of c) comprise 8-oxoguanine, wherein the method further comprises subjecting the product of strand extension to polymerase chain reaction (PCR) amplification after strand extension in c), and wherein the sequencing comprises identifying a guanine→thymine mutation introduced by PCR corresponding to a position of an adenine nucleotide base-paired with an 8-oxoguanine during strand extension.

The disclosures of all publications, patents, and patent applications referred to herein are each hereby incorporated by reference in their entireties. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Testing 3′ End Capture Adapters on Synthetic Templates

This Example demonstrates the utility of 3′ end capture adapters of the present disclosure in capturing different types of DNA templates, including a dsDNA fragment with 3′ overhangs, a blunt-ended dsDNA fragment, and a ssDNA fragment.

Three synthetic DNA fragments were constructed from single-stranded oligos as templates. First, an 83 bp dsDNA template with 10 bp 3′ overhangs was generated using oligos having the sequences of SEQ ID Nos: 3 & 4, respectively (FIG. 6B). Degradation of the overhangs would result in a 63 bp dsDNA fragment. Second, a blunt 90 bp dsDNA template with no overhangs was generated using oligos having the sequences of SEQ ID Nos: 5 & 6, respectively (FIG. 6C). Third, a 72 bp ssDNA template was generated as an oligo with SEQ ID NO: 7 (FIG. 6D). The sequences of the 3′ end capture adapter are shown in FIG. 6A (first and second strands having the sequences of SEQ ID Nos: 1 & 2, respectively). Sequences used in this example are shown in Table 1 below.

TABLE 1
Oligonucleotide sequences (5′ to 3′)

| | | SEQ |
|---|---|---|
| Sequence | | ID |
| type | Sequence | NO |
| 3′ end | GATCGGAAGAGCACGTCTGA | 1 |
| capture | ACTCC*A*G*T*/3ddC/ | |
| adapter | | |
| first | | |
| strand | | |
| 3′ end | ACACTCTTTCCCTACACGAC | 2 |
| capture | TCTTCCGATC*I*I*A | |
| adapter | | |
| second | | |
| strand | | |
| 3′ | GAATCTGGCGGACCCGGCTTA | 3 |
| overhang | CCGCCGCCGTCGCATCATCAA | |
| dsDNA | ACGGCGTCAGCCGATCTGCTC | |
| | AGTTGCATCC | |
| | GAGCAGATCGGCTGACGCCGT | 4 |
| | TTGATGATGCGACGGCGGCGG | |
| | TAAGCCGGGTCCGCCAGATTC | |
| | GGTGCTCTCC | |
| Blunt | GGAGCTGAAGCCATCCGGGCA | 5 |
| dsDNA | CTGGCCACACACGCGTGGTTG | |
| | GCAGGGCTTGGAAAAAGCATT | |
| | TGTAAAGAAAGAAATCGACCG | |
| | GGCTCC | |
| | GGAGCCCGGTCGATTTCTTTC | 6 |
| | TTTACAAATGCTTTTTCCAAG | |
| | CCCTGCCAACCACGCGTGTGT | |
| | GGCCAGTGCCCGGATGGCTTC | |
| | AGCTCC | |
| SSDNA | GTTATCAGGTCATGAACGGCA | 7 |
| | TCGCCGTGCCCGTTCAATCTG | |
| | TCTGATGTATCCATATTGGAT | |
| | CTGGTATCC | |

* indicates phosphorothioate linkage
I indicates inosine
3ddC indicates 3′ dideoxycytosine

The 3′ end capture adapter described above and a standard library prep adapter (NEBNext® Ultra™ II; NEB) were used to capture each template under standard library prep conditions. It was hypothesized that the 3′ end capture adapter would capture the dsDNA with 3′ overhangs with overhangs intact, the standard adapter would capture the dsDNA with 3′ overhangs (but degrading the overhangs) and the blunt-ended dsDNA, and neither would capture SSDNA.

Briefly, template oligos were annealed in buffer (95° C. for 5 minutes, followed by slow ramp down to 25° C.) and aliquoted at 20 ng/sample. Each template went through both of the following workflows and were not mixed together. For 3′ end capture adapters, adapter and oligos were incubated with T4 ligase and PNK at 16° C. overnight, then dNTPs and T4 DNA polymerase were added and mixtures incubated at 12° C. for 15 minutes. Samples were then subjected to standard cleanup (AMPure; Beckman Coulter), PCR with EM-Seq primers for 10 cycles, standard cleanup (AMPure; Beckman Coulter), then fluorometric quantification (Qubit; Thermo Fisher) and electrophoresis (TapeStation; Agilent). For standard adapters, adapter and oligos were subjected to Ultra II end repair (20° C. for 30 minutes→65° C. for 30 minutes), then Ultra II ligase mix was added and mixtures incubated at 20° C. for 15 minutes. Samples were then subjected to standard cleanup (AMPure; Beckman Coulter), PCR with EM-Seq primers for 10 cycles, standard cleanup (AMPure; Beckman Coulter), then fluorometric quantification (Qubit; Thermo Fisher) and electrophoresis (TapeStation; Agilent).

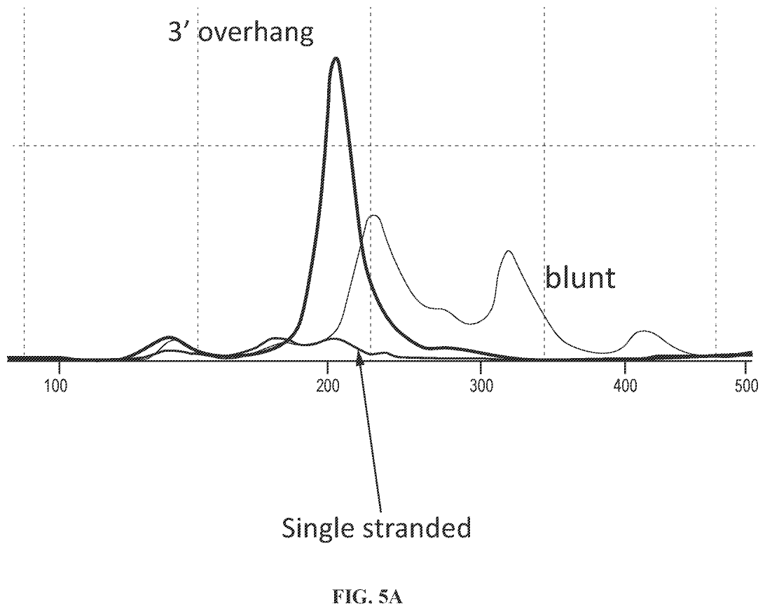

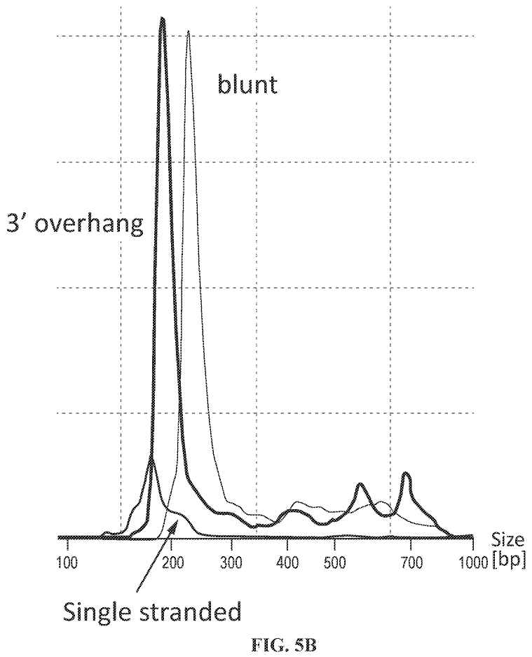

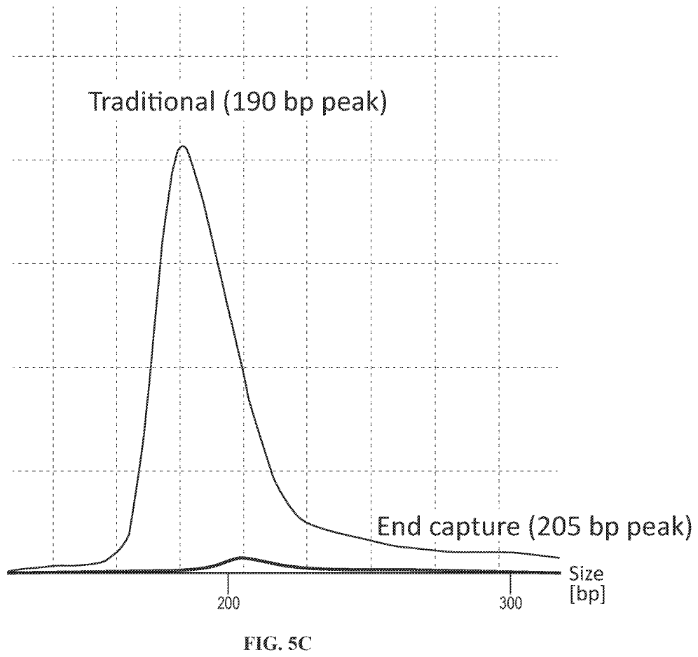

The results are shown in FIG. 5A for 3′ end capture adapters and FIG. 5B for standard adapters. As shown in FIG. 5A, the 3′ end capture adapters gave a single peak corresponding to the 3′ overhang template, showing selectivity for 3′ overhangs. In contrast, traditional adapters captured both dsDNA fragments (blunt-ended and with 3′ overhangs; FIG. 5B). Displaying only 3′ overhang peaks on a single graph (FIG. 5C) reveals that the peak obtained from traditional adapters was left-shifted compared to the peak obtained from 3′ end capture adapters. This suggests a smaller product, which is consistent with traditional adapters leading to degradation of 3′ overhangs, while 3′ end capture adapters preserving the 3′ overhangs. Thus, traditional adapters capture the template, but degrade 3′ overhangs in the process, resulting in a product that is ~15 bp smaller than that captured by 3′ end capture adapters, which do not result in 3′ overhang degradation.

What is claimed is:

1. A method for appending an oligonucleotide adapter to a single-stranded DNA (ssDNA) fragment or double-stranded DNA (dsDNA) fragment comprising 3′ overhang(s), the method comprising:
a) contacting an oligonucleotide adapter with a plurality of ssDNA fragments and/or dsDNA fragments comprising 3′ overhang(s) under conditions suitable for hybridization of the adapter with a ssDNA or dsDNA fragment of the plurality, wherein the adapter comprises:
a first strand comprising, from 5′ to 3′: a first portion comprising one or more nucleotides, and a second portion comprising three or more consecutive nucleotides joined by phosphorothioate linkages followed by a 3′ terminal dideoxynucleotide; and
a second strand comprising, from 5′ to 3′: a first portion comprising one or more nucleotides, and a second portion comprising at least two deoxyinosine nucleotides (dITPs) joined by a phosphorothioate linkage followed by a 3′ terminal deoxynucleotide;
wherein the first portion of the second strand of the adapter hybridizes with the first portion of the first strand of the adapter; and
wherein the second portion of the second strand of the adapter hybridizes with a 3′ end of the ssDNA fragment of the plurality or a 3′ overhang of the dsDNA fragment of the plurality;
b) incubating the hybridized adapter and ssDNA or dsDNA fragment with a DNA ligase under conditions suitable for sealing a nick between a 3′ end of the ssDNA fragment or overhang of the dsDNA fragment and the 5′ end of the first portion of the first strand of the adapter; and
c) incubating the hybridized adapter and fragment with a DNA polymerase and nucleotides under conditions suitable for the polymerase to catalyze strand extension from the 3′ terminal deoxynucleotide of the second strand of the adapter using the ssDNA fragment or overhang of the dsDNA fragment as a template, wherein the DNA polymerase lacks strand displacement activity, thereby appending the adapter onto the fragment.

2. The method of claim 1, wherein b) comprises incubating the hybridized adapter and ssDNA or dsDNA fragment with the DNA ligase and a polynucleotide kinase (PNK) under conditions suitable for sealing a nick between a 3′ end of the ssDNA fragment or overhang of the dsDNA fragment and the 5′ end of the first portion of the first strand of the adapter.

3. The method of claim 1, wherein the adapter is hybridized with a dsDNA fragment comprising a 3′ overhang, and the method further comprises, after strand extension in c), incubating the hybridized adapter and fragment with a DNA ligase under conditions suitable for sealing a nick between the strand synthesized in c) and a 5′ end of the dsDNA fragment.

4. The method of claim 3, wherein the method further comprises, after strand extension in c), incubating the hybridized adapter and fragment with the DNA ligase and a PNK under conditions suitable for sealing a nick between the strand synthesized in c) and a 5′ end of the dsDNA fragment.

5. The method of claim 1, wherein the 3′ terminal deoxynucleotide of the second strand is a deoxyadenosine trisphosphate (dATP), deoxycytidine triphosphate (dCTP), deoxyguanosine triphosphate (dGTP), or deoxythymidine triphosphate (dTTP).

6. The method of claim 1, wherein:
(a) the one or more nucleotides of the first portions of the first and second strands are deoxynucleotide triphosphate (dNTP) nucleotides;
(b) the first portion of the first strand and the first portion of the second strand each comprise 10 or more nucleotides; and/or
(c) the second portion of the second strand comprises at least two consecutive dITPs joined by a phosphorothioate linkage followed by the 3′ terminal deoxynucleotide; wherein optionally the second portion of the second strand comprises two consecutive dITPs joined by a phosphorothioate linkage followed by the 3′ terminal deoxynucleotide.

7. The method of claim 1, wherein the second strand further comprises a third portion comprising one or more nucleotides, wherein the third portion is 5′ of the first portion, and wherein, upon hybridization of the first portion of the first strand with the first portion of the second strand, the third portion of the second strand does not hybridize with the second portion of the first strand.

8. The method of claim 1, wherein the DNA polymerase is a T4 DNA polymerase and/or the DNA ligase is a T3 DNA ligase, T4 DNA ligase, or Taq DNA ligase.

9. The method of claim 1, wherein the plurality of ssDNA fragments and/or dsDNA fragments comprising 3′ overhang(s) are contacted in a) with a plurality of adapters that comprises a first adapter with deoxyadenosine trisphosphate (dATP) as the 3′ terminal deoxynucleotide of the second strand, a second adapter with deoxycytidine triphosphate (dCTP) as the 3′ terminal deoxynucleotide of the second strand, a third adapter with deoxyguanosine triphosphate (dGTP) as the 3′ terminal deoxynucleotide of the second strand, and a fourth adapter with deoxythymidine triphosphate (dTTP) as the 3′ terminal deoxynucleotide of the second strand.

10. The method of claim 1, wherein the nucleotides of c) comprise at least one modified nucleotide; wherein optionally the modified nucleotide is 8-oxoguanine, 5-methylcytosine (5mC), or inosine.

11. The method of claim 1, further comprising, prior to a), isolating the plurality of ssDNA fragments and/or dsDNA fragments comprising 3′ overhang(s) from a sample; wherein optionally:
(a) the sample comprises cell-free DNA (cfDNA), circulating cell-free DNA (ccfDNA), or circulating tumor DNA (ctDNA);
(b) the sample comprises fluid, cells, or tissue; or
(c) the sample comprises tumor cells and/or tumor nucleic acids.

12. The method of claim 11, wherein the sample further comprises blunt-ended dsDNA fragments and/or dsDNA fragments comprising 5′ overhang(s), and wherein the method further comprises, prior to a), isolating the blunt-ended dsDNA fragments and/or dsDNA fragments comprising 5′ overhang(s) with the plurality of ssDNA fragments and/or dsDNA fragments comprising 3′ overhang(s).

13. The method of claim 12, further comprising, after strand extension in c):
(i) subjecting the blunt-ended dsDNA fragments and/or dsDNA fragments comprising 5′ overhang(s) to end repair;
(ii) subjecting the end-repaired dsDNA fragments to 3′ adenylation; and
(iii) subjecting the 3′ adenylated dsDNA fragments to adapter ligation;
wherein (i)-(iii) are optionally performed in the presence of the product of strand extension from c).

14. The method of claim 1, further comprising, after strand extension in c):
(a) isolating the product of strand extension from free oligonucleotide adapters;
(b) subjecting the product of strand extension to polymerase chain reaction (PCR) amplification; and/or
(c) subjecting the product of strand extension or PCR amplicon(s) thereof to sequencing or methyl sequencing.

15. The method of claim 14, wherein the method comprises subjecting the product of strand extension or PCR amplicon(s) thereof to sequencing or methyl sequencing after strand extension in c); wherein the nucleotides of c) comprise 8-oxoguanine; and wherein the method further comprises subjecting the product of strand extension to polymerase chain reaction (PCR) amplification after strand extension in c), wherein the sequencing comprises identifying a guanine→thymine mutation introduced by PCR corresponding to a position of an adenine nucleotide base-paired with an 8-oxoguanine during strand extension.

16. An oligonucleotide, comprising:
a) a first strand that comprises, from 5′ to 3′:
i) a first portion comprising one or more nucleotides, and
ii) a second portion comprising three or more consecutive nucleotides joined by phosphorothioate linkages followed by a 3′ terminal dideoxynucleotide (ddNTP); and
b) a second strand that comprises, from 5′ to 3′:
i) a first portion comprising one or more nucleotides, wherein the first portion of the second strand is capable of hybridizing with the first portion of the first strand; and
ii) a second portion comprising at least two deoxyinosine nucleotides (dITPs) joined by a phosphorothioate linkage followed by a 3′ terminal deoxynucleotide;
wherein upon hybridization of the first portion of the first strand with the first portion of the second strand, the second portion of the second strand forms a 3′ overhang.

17. The oligonucleotide of claim 16, wherein the 3′ terminal deoxynucleotide of the second strand is a deoxyadenosine trisphosphate (dATP), deoxycytidine triphosphate (dCTP), deoxyguanosine triphosphate (dGTP), or deoxythymidine triphosphate (dTTP).

18. The oligonucleotide of claim 16, wherein:
(a) the one or more nucleotides of the first portions of the first and second strands are deoxynucleotide triphosphate (dNTP) nucleotides;
(b) the first portion of the first strand and the first portion of the second strand each independently comprise 10 or more nucleotides; and/or
(c) the second portion of the second strand comprises at least two consecutive dITPs joined by a phosphorothioate linkage followed by the 3′ terminal deoxynucleotide, wherein optionally the second portion of the second strand comprises two consecutive dITPs joined by a phosphorothioate linkage followed by the 3′ terminal deoxynucleotide.

19. The oligonucleotide of claim 16, wherein the second strand further comprises a third portion comprising one or more nucleotides, wherein the third portion is 5′ of the first portion, and wherein, upon hybridization of the first portion of the first strand with the first portion of the second strand, the third portion of the second strand does not hybridize with the second portion of the first strand.

20. A kit of oligonucleotides, comprising:
a) a first oligonucleotide according to claim 17, wherein the 3′ terminal deoxynucleotide of the second strand is a deoxyadenosine trisphosphate (dATP);
b) a second oligonucleotide according to claim 17, wherein the 3′ terminal deoxynucleotide of the second strand is a deoxycytidine triphosphate (dCTP);
c) a third oligonucleotide according to claim 17, wherein the 3′ terminal deoxynucleotide of the second strand is a deoxyguanosine triphosphate (dGTP); and
d) a fourth oligonucleotide according to claim 17, wherein the 3′ terminal deoxynucleotide of the second strand is a deoxythymidine triphosphate (dTTP).
